(19) United States
(12) Patent Application Publication
(10) Pub. No.: US 2025/0120706 A1
(43) Pub. Date: Apr. 17, 2025

(54) SURGICAL INSTRUMENTS WITH JAW AND FIRING ACTUATOR LOCKOUT ARRANGEMENTS LOCATED PROXIMAL TO A JAW PIVOT LOCATION

(71) Applicant: Cilag GmbH International, Zug (CH)

(72) Inventors: Frederick E. Shelton, IV, Hillsboro, OH (US); Jason L. Harris, Lebanon, OH (US)

(21) Appl. No.: 18/379,811

(22) Filed: Oct. 13, 2023

(51) Int. Cl. A61B 17/072

(52) U.S. Cl. CPC A61B 17/072; A61B 2017/07257

(57) ABSTRACT

A surgical stapling system is disclosed comprising an end effector, a closure actuator, and a closure lockout. The end effector comprises an elongate channel, an anvil, and a pivot pivotably coupling the anvil to the elongate channel. The anvil is pivotable relative to the elongate channel between an open position and a closed position. The closure actuator is movable from a first position toward a second position during a closure stroke. The closure actuator is configured to move the anvil toward the closed position. The closure lockout is configurable between a locked state in which the closure lockout engages the closure actuator at a location proximal with respect to the pivot to prevent the closure actuator from moving the anvil toward the closed position and an unlocked state in which the closure actuator is permitted to move the anvil toward the closed position.

### BACKGROUND

The present invention relates to surgical instruments and, in various arrangements, to surgical stapling and cutting instruments and staple cartridges for use therewith that are designed to staple and cut tissue.

### SUMMARY

In one form of the invention, lockouts are utilized with surgical stapling and cutting instruments to ensure that certain conditions are met prior to allowing a clinician to cut and staple tissue with the surgical stapling and cutting instrument. In one aspect, these lockouts ensure that patient tissue is not inadvertently cut and/or stapled prior to these conditions being met.

In accordance with the present disclosure, a surgical stapling system is disclosed comprising an end effector, a closure actuator, and a closure lockout. The end effector comprises an elongate channel, an anvil, and a pivot pivotably coupling the anvil to the elongate channel. The anvil is pivotable relative to the elongate channel between an open position and a closed position. The closure actuator is movable from a first position toward a second position during a closure stroke. The closure actuator is configured to move the anvil toward the closed position. The closure lockout is configurable between a locked state in which the closure lockout engages the closure actuator at a location proximal with respect to the pivot to prevent the closure actuator from moving the anvil toward the closed position and an unlocked state in which the closure actuator is permitted to move the anvil toward the closed position.

In accordance with the present disclosure, a surgical stapling system is disclosed comprising an end effector, a firing actuator, and a lockout. The end effector comprises an elongate channel, an anvil, and a pivot pivotably coupling the anvil to the elongate channel. The anvil is pivotable relative to the elongate channel between an open position and a closed position. The firing actuator is movable through a closure stroke and a firing stroke distal and subsequent to the closure stroke. The firing actuator is configured to move the anvil toward the closed position based on the firing actuator moving through the closure stroke, The firing actuator is configured to deploy staples from a staple cartridge positioned in the end effector based on the firing actuator moving through the firing stroke. The lockout is configurable between a locked state in which the lockout engages the firing actuator proximal with respect to the pivot to prevent the firing actuator from moving through the closure stroke and the firing stroke and an unlocked state in which firing actuator is allowed to move through the closure stroke and the firing stroke.

In accordance with the present disclosure, a surgical stapling system is disclosed comprising an end effector, a closure actuator, a firing actuator, a closure lockout, and a firing lockout. The end effector comprises an elongate channel, an anvil, and a pivot pivotably coupling the anvil to the elongate channel. The anvil is pivotable relative to the elongate channel between an open position and a closed position. The closure actuator is movable from a first position toward a second position during a closure stroke. The closure actuator is configured to move the anvil toward the closed position. The firing actuator is movable from an unfired position toward a fired position during a firing stroke. The firing actuator comprises a knife configured to cut tissue captured between the elongate channel and the anvil. The closure lockout is positioned proximal with respect to the pivot. The closure lockout is configurable between a first locked state in which the closure actuator is prevented from moving the anvil toward the closed position and a first unlocked state in which the closure actuator is permitted to move the anvil toward the closed position. The firing lockout is configurable between a second locked state in which the firing actuator is prevented from moving toward the fired position and a second unlocked state in which the firing actuator is permitted to move toward the fired position.

### LISTING OF THE FIGURES

Various features of the embodiments described herein, together with advantages thereof, may be understood in accordance with the following description taken in conjunction with the accompanying drawings as follows:

FIG. 1 is a partial perspective view of a surgical stapling system including an end effector;

FIG. 2 is a perspective view of the end effector of FIG. 1 with a staple cartridge removed therefrom;

FIG. 3 is another perspective view of the end effector of FIG. 1 with the staple cartridge removed;

FIG. 4 is an elevational view of the end effector of FIG. 1 in a closed position;

FIG. 5 is an elevational view of the end effector of FIG. 1 in an open position;

FIG. 6 is a perspective view of a surgical stapling system that includes a lockout;

FIG. 7 is a side elevational view of the surgical stapling system of FIG. 6 with the lockout in a locked state;

FIG. 8 is another side elevational view of the surgical stapling system of FIG. 6 with the lockout in an unlocked state;

FIG. 9 is a top view of a surgical stapling system with a compatible and ready-to-fire staple cartridge removably positioned in an end effector with two lockouts in their respective unlocked states;

FIG. 10 is the top of the surgical stapling system of FIG. 9 without a staple cartridge removably positioned in the end effector and the two lockouts in their respective locked states;

FIG. 11 is a partial perspective view of the surgical stapling system of FIG. 10;

FIG. 12 illustrates a surgical stapling system that includes a closure lockout;

FIG. 13 is a partial perspective view of a surgical stapling system that includes a lockout that has been placed into an unlocked state by a compatible staple cartridge;

FIG. 14 is a partial perspective view of the surgical stapling system of FIG. 13 with the lockout in a locked state; and

FIG. 15 is a partial perspective view of the surgical stapling system of FIG. 13 with the lockout in another locked state.

Corresponding reference characters indicate corresponding parts throughout the several views.

### DETAILED DESCRIPTION

Applicant of the present application owns the following U.S. patent applications that were filed on even date herewith and which are each herein incorporated by reference in their respective entireties:
U.S. patent application, titled METHOD OF OPERATING A SURGICAL STAPLING INSTRUMENT; Attorney Docket No. END9484USNP2/220491-2M;
U.S. patent application, titled SURGICAL STAPLING SYSTEMS WITH ADAPTIVE STAPLE FIRING ALGORITHMS; Attorney Docket No. END9484USNP3/220491-3;
U.S. patent application, titled LEARNED TRIGGERS FOR ADAPTIVE CONTROL OF SURGICAL STAPLING SYSTEMS; Attorney Docket No. END9484USNP4/220491-4;
U.S. patent application, titled CONTROL CIRCUIT FOR ACTUATING MOTORIZED FUNCTION OF SURGICAL STAPLING INSTRUMENT UTILIZING INERTIAL DRIVE TRAIN PROPERTIES; Attorney Docket No. END9484USNP5/220491-5;
U.S. patent application, titled PROPORTIONATE BALANCING OF THE FUNCTION IMPACT MAGNITUDE OF BATTERY OUTPUT TO PEAK MOTOR CURRENT; Attorney Docket No. END9484USNP6/220491-6;
U.S. patent application, titled MOTOR OPTIMIZATION BY MINIMIZATION OF PARASITIC LOSSES AND TUNING MOTOR DRIVE CONFIGURATION; Attorney Docket No. END9484USNP7/220491-7;
U.S. patent application, titled APPARATUS AND METHOD TO REDUCE PARASITIC LOSSES OF THE ELECTRICAL SYSTEM OF A SURGICAL INSTRUMENT; Attorney Docket No. END9484USNP8/220491-8;
U.S. patent application, titled SURGICAL TOOL WITH RELAXED FLEX CIRCUIT ARTICULATION; Attorney Docket No. END9484USNP9/220491-9;
U.S. patent application, titled WIRING HARNESS FOR SMART STAPLER WITH MULTI AXIS ARTICULATION; Attorney Docket No. END9484USNP10/220491-10;
U.S. patent application, titled SURGICAL SYSTEM WITH WIRELESS ARRAY FOR POWER AND DATA TRANSFER; Attorney Docket No. END9484USNP11/220491-11; and
U.S. patent application, titled SURGICAL STAPLE CARTRIDGE COMPRISING REPLACEABLE ELECTRONICS PACKAGE; Attorney Docket No. END9484USNP12/220491-12.

Applicant of the present application owns the following U.S. patent applications that were filed on even date herewith and which are each herein incorporated by reference in their respective entireties:
U.S. patent application, titled METHOD OF ASSEMBLING A STAPLE CARTRIDGE; Attorney Docket No. END9484USNP13/220491-13M;
U.S. patent application, titled CONTROL SURFACES ON A STAPLE DRIVER OF A SURGICAL STAPLE CARTRIDGE; Attorney Docket No. END9484USNP14/220491-14;
U.S. patent application, titled INTEGRAL CARTRIDGE STIFFENING FEATURES TO REDUCE CARTRIDGE DEFLECTION; Attorney Docket No. END9484USNP15/220491-15;
U.S. patent application, titled STAPLE CARTRIDGE COMPRISING WALL STRUCTURES TO REDUCE CARTRIDGE DEFLECTION; Attorney Docket No. END9484USNP16/220491-16;
U.S. patent application, titled PAN-LESS STAPLE CARTRIDGE ASSEMBLY COMPRISING RETENTION FEATURES FOR HOLDING STAPLE DRIVERS AND SLED; Attorney Docket No. END9484USNP17/220491-17;
U.S. patent application, titled STAPLE CARTRIDGE COMPRISING A SLED HAVING A DRIVER LIFT CAM; Attorney Docket No. END9484USNP18/220491-18;
U.S. patent application, titled SURGICAL STAPLE CARTRIDGES WITH SLEDS CONFIGURED TO BE COUPLED TO A FIRING DRIVER OF A COMPATIBLE SURGICAL STAPLER; Attorney Docket No. END9484USNP19/220491-19;
U.S. patent application, titled STAPLE CARTRIDGE COMPRISING A COMPOSITE SLED; Attorney Docket No. END9484USNP20/220491-20;
U.S. patent application, titled SURGICAL INSTRUMENTS WITH LATERALLY ENGAGEABLE LOCKING ARRANGEMENTS FOR LOCKING A FIRING ACTUATOR; Attorney Docket No. END9484USNP22/220491-22;
U.S. patent application, titled DUAL INDEPENDENT KEYED LOCKING MEMBERS ACTING ON THE SAME DRIVE MEMBER; Attorney Docket No. END9484USNP23/220491-23;
U.S. patent application, titled ADJUNCTS FOR USE WITH SURGICAL STAPLING INSTRUMENTS; Attorney Docket No. END9484USNP24/220491-24;
U.S. patent application, titled ADJUNCTS FOR USE WITH SURGICAL STAPLING INSTRUMENTS; Attorney Docket No. END9484USNP25/220491-25;
U.S. patent application, titled JAW CONTROL SURFACES ON A SURGICAL INSTRUMENT JAW; Attorney Docket No. END9484USNP26/220491-26;
U.S. patent application, titled ZONED ALGORITHM ADAPTIVE CHANGES BASED ON CORRELATION OF COOPERATIVE COMPRESSION CONTRIBUTIONS OF TISSUE; Attorney Docket No. END9484USNP27/220491-27;
U.S. patent application, titled STAPLE CARTRIDGES COMPRISING TRACE RETENTION FEATURES; Attorney Docket No. END9484USNP29/220491-29; and.
U.S. patent application, titled STAPLE CARTRIDGES COMPRISING STAPLE RETENTION FEATURES; Attorney Docket No. END9484USNP30/220491-30.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working frame through which the end effector and elongate shaft of a surgical instrument can be advanced.

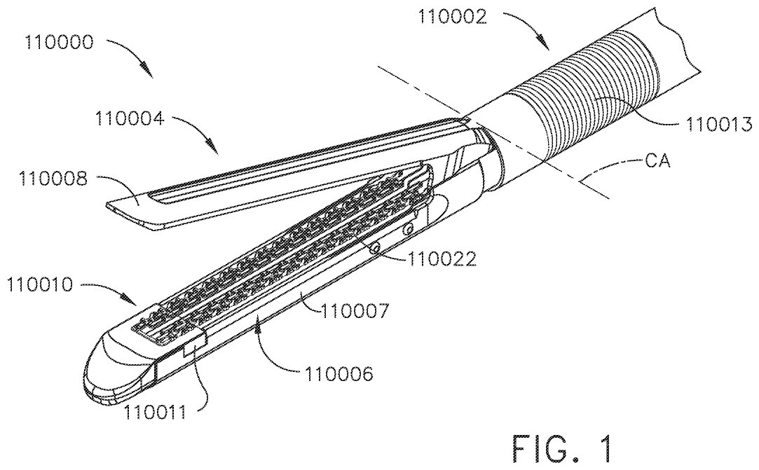

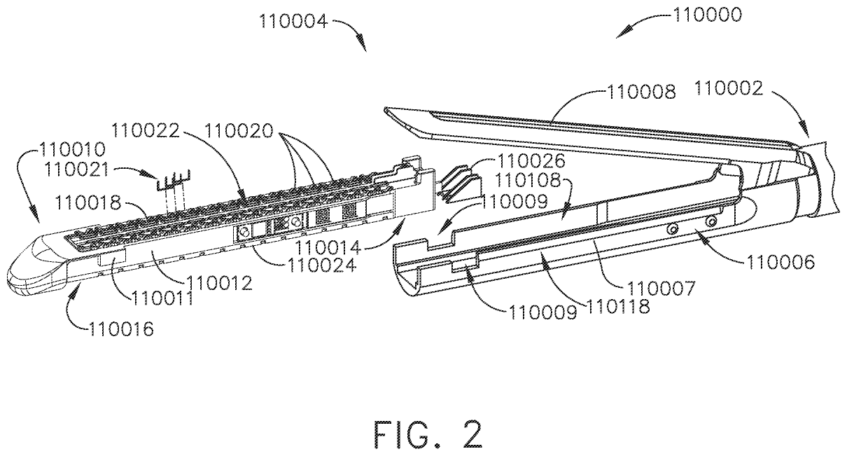

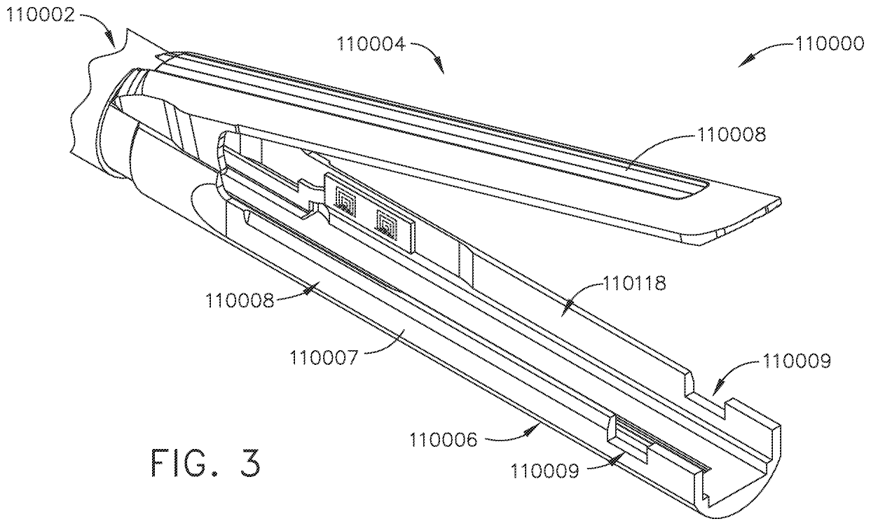

Referring now to FIGS. 1-5, a surgical stapling system 110000 is shown that comprises a shaft 110002 and an end effector 110004 extending from the shaft 110002. The end effector 110004 comprises a first jaw 110006 and a second jaw 110008. The first jaw 110006 comprises an elongate channel 110007 and a staple cartridge 110010. The staple cartridge 110010 is insertable into and removable from the elongate channel 110007. In particular, the staple cartridge 110010 comprises tabs 110011 on each lateral side thereof that snap into corresponding recesses 110009 defined in the elongate channel 110007. Alternatively, the staple cartridge 110010 may not be removable from, or at least readily replaceable from, the first jaw 110006.

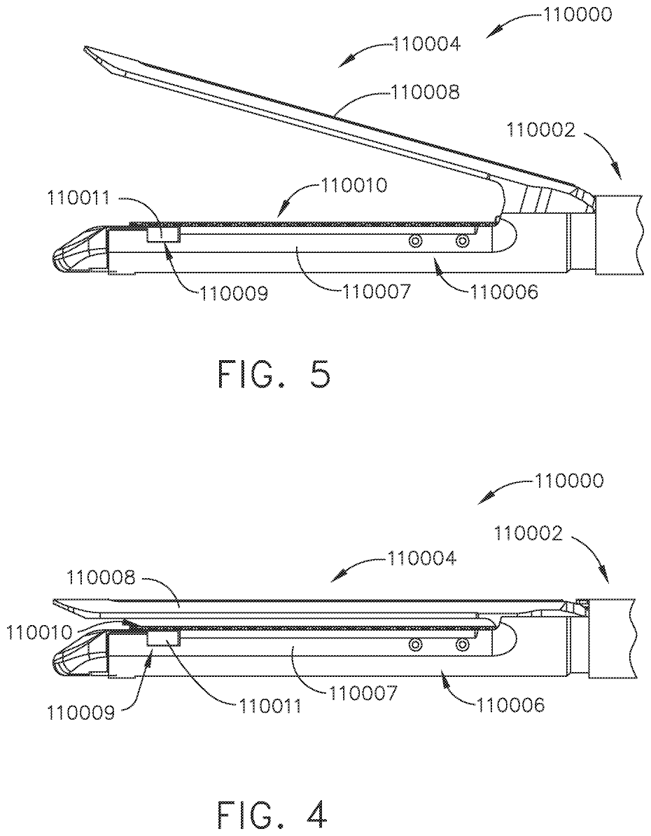

The second jaw 110008 comprises an anvil configured to deform staples ejected from the staple cartridge 110010. The second jaw 110008 is pivotable relative to the first jaw 11006 about a closure axis CA between an open position (FIG. 5) and a closed position (FIG. 4); however, the first jaw 110006 may be pivotable relative to the second jaw 110008. The surgical stapling system 110000 further comprises an articulation joint 110013 configured to permit the end effector 110004 to be rotated, or articulated, relative to the shaft 110002. Alternatively, the surgical stapling system 11000 may not include an articulation joint.

The staple cartridge 110010 comprises a cartridge body 110012. The cartridge body 110012 includes a proximal end 110014, a distal end 110016, and a deck 110018 extending between the proximal end 110014 and the distal end 110016. In use, the staple cartridge 110010 is positioned on a first side of the tissue to be stapled and the anvil 110008 is positioned on a second side of the tissue. The anvil 110008 is moved toward the staple cartridge 110010 (see FIGS. 4 and 5) to compress and clamp the tissue against the deck 110018. Thereafter, staples 110021 removably stored in the cartridge body 110012 can be deployed into the tissue. The cartridge body 110012 includes staple cavities 110020 defined therein wherein staples are removably stored in the staple cavities 110020. The staple cavities 110020 are arranged in six longitudinal rows. Three rows of staple cavities are positioned on a first side of a longitudinal slot 110022 and three rows of staple cavities are positioned on a second side of the longitudinal slot 110022. Other arrangements of staple cavities and staples may be possible.

The staples are supported by staple drivers in the cartridge body 110012. Staples supported on staple drivers can be seen in U.S. Patent Application Publication No. 2021/0059672, entitled SURGICAL INSTRUMENT WITH WIRELESS COMMUNICATION BETWEEN A CONTROL UNIT OF A ROBOTIC SYSTEM AND REMOTE SENSOR, filed Sep. 14, 2020, which is hereby incorporated by reference in its entirety herein. The drivers are movable between a first, unfired position, and a second, fired, position to eject the staples from the staple cavities 110020. The drivers are retained in the cartridge body 110012 by a retainer 110024 which extends around the bottom of the cartridge body 110012 and includes resilient members configured to grip the cartridge body 110012 and hold the retainer 110024 to the cartridge body 110012. The drivers are movable between their unfired positions and their fired positions by a sled 110026. The sled 110026 is movable between a proximal position adjacent the proximal end 110014 and a distal position adjacent the distal end 110016. The sled 110026 comprises a plurality of ramped surfaces configured to slide under the drivers and lift the drivers, and the staples supported thereon, toward the anvil 110008. Alternatively, the staples may not be supported by staple drivers, but rather, the staples may be include integral drive surfaces that are directly engaged by the sled 110026 to lift the staples, examples of which are described in U.S. Patent Application Publication No. 2015/0173756, entitled SURGICAL CUTTING AND STAPLING METHODS, filed Dec. 23, 2013, which is hereby incorporated by reference in its entirety herein.

Further to the above, the sled 110026 is moved distally by a firing member or firing actuator, such as those seen and described elsewhere herein. The firing actuator is configured to contact the sled 110026 and push the sled 110026 toward the distal end 110016. The longitudinal slot 110022 defined in the cartridge body 110012 is configured to receive the firing actuator. The anvil 110008 also includes a slot defined therein that receives the firing actuator. The firing actuator further comprises a first cam which engages the first jaw 110006 and a second cam which engages the second jaw 110008 in the slot of the anvil. As the firing actuator is advanced distally, the first cam and the second cam can control the distance, or tissue gap, between the deck 110018 of the staple cartridge 110010 and the anvil 110008. The firing actuator also comprises a knife configured to incise the tissue captured intermediate the staple cartridge 110010 and the anvil 110008. It is desirable for the knife to be positioned at least partially proximal to the ramped surfaces such that the staples are ejected ahead of the knife.

In accordance with the present disclosure, the anvil 110008 may moved from the open position to the closed position using a closure actuator that is controlled separately from the firing actuator. As such, the firing actuator can be considered to be a part of a firing system that is separate and distinctly operable from the closure actuator. The anvil 110008 may comprise a ramp on a proximal end thereof and the closure actuator comprises a closure tube that movable distally to engage the ramp and cam the anvil 110008 to the closed position. In the closed position, the first cam and the second cam of the firing actuator translate distally and maintain the anvil 110008 in the closed position. In such arrangements, to transition the anvil 110008 to the open position, the closure tube is retracted proximal and springs positioned within the end effector 110004 bias the anvil 110008 to the open position. The anvil 110008 can include a tab and the closure tube defines an aperture which engages the tab as the closure tube moves proximally, thereby positively transitioning the anvil 110008 to the open position. Exemplary closure actuators and closure tubes are described in U.S. Patent Application Publication No. 2021/0059672, entitled SURGICAL INSTRUMENT WITH WIRELESS COMMUNICATION BETWEEN A CONTROL UNIT OF A ROBOTIC SYSTEM AND REMOTE SENSOR, filed Sep. 14, 2020, which is hereby incorporated by reference in its entirety herein. Various other closure actuators are contemplated by the present disclosure and are discussed elsewhere herein.

The anvil 110008 can be moved from the open position to the closed position using the firing actuator. As such, the anvil 110008 may include a ramp that extends proximally from walls that define the slot that is engaged by the firing actuator as it moves from a first, proximal to position to a second, intermediate position during a first portion of the stroke of the firing actuator to move the anvil 110008 to the closed position. The first portion of the stroke of the firing actuator can be about 0.110″-0.250″ of the firing actuator travel. At the end of the first portion of the stroke, the firing actuator can continue advancing distally from the second, intermediate position toward a third, distal position during a second portion of the stroke to deploy staples from the staple cartridge 110010 and incise tissue captured by the end effector, as described above. Exemplary firing actuators that close the anvil and fire staples are described in U.S. Pat. No. 11,160,551, entitled ARTICULATABLE SURGICAL STAPLING INSTRUMENTS, filed Dec. 21, 2016, which is hereby incorporated by reference in its entirety herein. When a firing actuator is utilized to move the anvil 110008 toward the closed position and deploy staples from the staple cartridge 110010, the firing actuator can be considered both a closure actuator and/or a firing actuator.

After a staple cartridge 110010 has been fired, or at least partially fired, it is removed from the elongate channel 110007 and then replaced with another replaceable staple cartridge, if desired. At such point, the surgical stapling system 110000 can be re-used to continue stapling and incising the patient tissue. However, a previously-fired staple cartridge can be accidentally loaded into the elongate channel 110007. If the firing actuator were to be advanced distally within such a previously-fired staple cartridge, the surgical stapling system 110000 could cut the patient tissue without stapling it. The surgical stapling system 110000 could similarly cut the patient tissue without stapling it if the firing actuator were advanced distally through a staple firing stroke without a staple cartridge positioned in the elongate channel 110007 at all. In addition, various surgical staple cartridges may have different arrays of and/or orientations of staples/fasteners therein. The sizes of the staples or fasteners, as well as the number of fasteners may vary from cartridge type to cartridge type depending upon a particular surgical procedure or application. To ensure that the staples are properly crimped or formed, the surgical staple cartridges must be used in connection with corresponding, compatible anvils that have the proper array of staple-forming pockets therein as well as the proper cutting and firing components. Should a “non-compatible” cartridge be loaded into a surgical stapling device that has an anvil that is mismatched to the staple cartridge, the staples may not be properly formed during the firing process which could lead to catastrophic results.

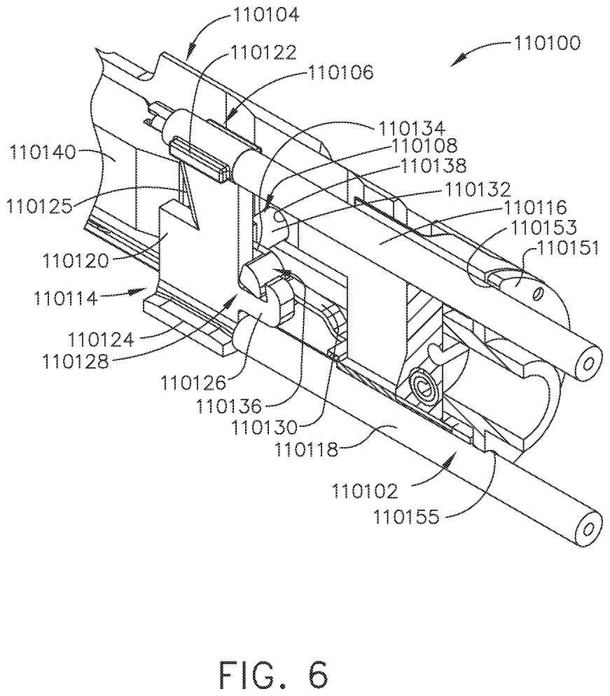

Referring now to FIGS. 6-8, a surgical stapling system 110100 is provided, in accordance with the present disclosure. The surgical stapling system 110100 comprises a shaft 110102, an end effector 110104, a firing actuator 110106, and a lockout 110108. The shaft 110102 can be similar to shaft 110002. The end effector 110104 can be similar to end effector 110004.

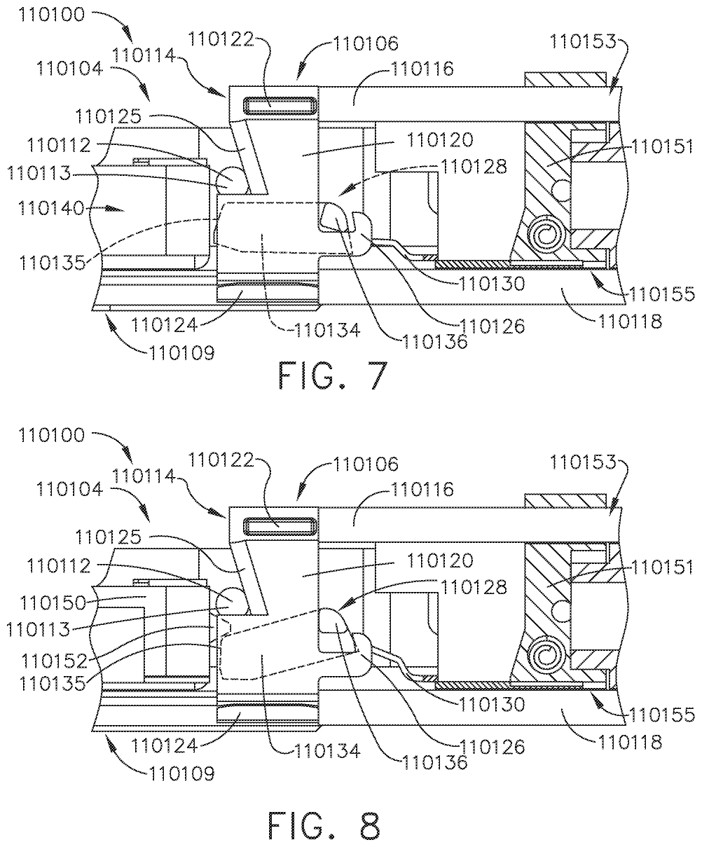

The end effector 110104 comprises an elongate channel 110109, an anvil (not shown, but similar to anvil 110008), and a pivot 110112 pivotably coupling the anvil to the elongate channel 110109 such that the anvil can rotate relative to the elongate channel 110109 between an open position (similar to FIG. 5) and a closed position (similar to FIG. 4). As can be seen in FIGS. 7 and 8, the pivot 110112 comprises a rod 110113 that defines a closure axis (similar to closure axis CA shown in FIG. 1).

The firing actuator 110106 comprises a firing driver 110114, a first push rod 110116 attached at an upper end of the firing driver 110114, and a second push rod 110118 attached at a lower end of the firing driver 110114. The first push rod 110116 and the second push rod 110118 can be driven using any suitable drive system, such as a motor drive system, to drive the firing driver 110114 from a proximal position, shown in FIGS. 7 and 8, to a distal position. An exemplary motor drive system is described in U.S. Patent Application Publication No. 2015/0173756, entitled SURGICAL CUTTING AND STAPLING METHODS, filed Dec. 23, 2013, which is hereby incorporated by reference in its entirety herein. The firing actuator comprises a body 110120, a first cam 110122, a second cam 110124, a knife 110125, and a hook 110126, described in more detail below.

In operation, the firing driver 110114 is movable through a stroke that includes a closure stroke portion and a firing stroke portion distal and subsequent to the closure stroke portion. During the closure stroke portion, the first cam 110122 of the firing driver 110114 engages a ramp on the anvil to cam the anvil from the open position toward the closed position. During the firing stroke portion subsequent and distal to the closure stroke portion, the body 110120 of the firing driver 110114 engages and drives a sled (similar to sled 110026) through a staple cartridge to deploy staples therefrom while the knife 110125 cuts the stapled tissue. Furthermore, as the firing driver 110114 is advanced distally through the firing stroke portion, the second cam 110124 engages the elongate channel 110109 and the first cam 110122 engages the anvil to control the distance, or tissue gap, therebetween. Since the firing actuator 110106 is utilized to move the anvil toward the closed position prior to deploying staples from a staple cartridge, the firing actuator can also be considered a closure actuator.

The surgical stapling system 110100 further comprises a flange 110151 that defines a first channel 110153 that receives and guides the first push rod 110116 and a second channel 110155 that receives and guides the second push rod 110118 during the stroke of the firing actuator.

The lockout 110108 includes lockout bar 110128 and a spring 110130 coupled to the lockout bar 110128. The lockout bar 110128 includes a pin 110132, a body 110134 defining a cam surface 110135 on its distal end thereof, and a tab 110136. The shaft 110102 defines an aperture 110138 that receives the pin 110132 of the lockout bar 110128. See FIG. 8. In accordance with the present disclosure, the elongate channel 110109 can define the aperture 110138 that receives the pin 110132 of the lockout bar 110128. The lockout bar 110128 is pivotable between a locked state in which the tab 110136 is received within the hook 110126 (FIGS. 6 and 7) and an unlocked state in which the tab 110136 is rotated out of, or displaced from, the hook 110126 (FIG. 8). The spring 110130 is mounted to the shaft 110102 and biases lockout bar 110128 toward the locked state. The spring 110130 can also be mounted to the base of the elongate channel 110109. The spring 110130 may comprise a leaf spring. The spring may comprise a torsion spring.

In the locked state, the tab 110136 and the hook 110126 are longitudinally aligned such that, should a user attempt to drive the firing driver 110114 through its stroke portions, the tab 110136 would engage the hook 110126, preventing distal motion of the firing driver 110114. Stated another way, in the locked state, the lockout 110108 prevents the firing driver 110114 from moving through the closure stroke portion and the firing stroke portion of its stroke. Notably, the tab 110136 engages the hook 110126 at a location that is proximal to the pivot 110112, as will be explained in more detail below.

In the unlocked state, the tab 110136 is rotated out of, or displaced from, the hook 110126 such that the tab 110136 and hook 110126 are not longitudinally aligned. Stated another way, in the unlocked state, the lockout 110108 allows the firing driver 110114 to move through the closure stroke portion and the firing stroke portion of its stroke. In one aspect, since the lockout 110108 prevents the firing driver 110114 from moving through the closure stroke portion and the firing stroke portion when it is in the locked state, the lockout 110108 can be considered a closure lockout and/or a firing lockout.

The majority, if not all, of the lockout 110108 is positioned proximal to the pivot 110112. In the illustrated arrangement, a majority of the lockout bar 110128 is located proximal to the pivot 110112. For example, the cam surface 110135 on the distal end of the lockout bar 110128 may not be proximal to the pivot 110112, however, the point of contact between the firing actuator 110106 (the hook 110126) and the lockout 110108 (the tab 110136) is proximal with respect to the pivot 110112, as described above. By moving the lockout 110108 proximal of the pivot 110112, space is freed up within the end effector 110104 and the lockout 110108 is moved away from the pinch point between the elongate channel 110109 and the anvil. The entirety of the lockout 110108, such as the cam surface 110135, may be positioned proximal to the pivot 110112, freeing up additional space within the end effector 110104.

To transition the lockout 110108 from the locked state to the unlocked state, a compatible staple cartridge is positioned in the elongate channel 110109. Referring to FIGS. 6 and 7, an incompatible staple cartridge 110140 is removably positioned in the elongate channel 110109. Accordingly, the lockout 110108 remains in the locked state, preventing the firing driver 110114 from moving through the closure stroke portion and the firing stroke portion of its stroke. Notably, the incompatible staple cartridge 110140 does not include a key to transition the lockout 110108 to the unlocked state, as described in more detail below. Accordingly, should a user attempt to drive the firing actuator 110106 through its strokes, the tab 110136 would engage the hook 110126 proximal of the pivot 110112, preventing the firing actuator 110106 from moving distally.

Referring now to FIG. 8, a compatible staple cartridge 110150 is removably positioned in the elongate channel 110109. The compatible staple cartridge 110150 includes a key 110152 extending proximally therefrom. As the staple cartridge 110150 is removably positioned in the elongate channel 110109, the key 110152 engages the cam surface 110135 of the lockout bar 110128. As the staple cartridge 110150 is moved proximally to seat the staple cartridge 110150 in the elongate channel 110109, the key 110152 engages the cam surface 110135 and rotates the tab 110136 out of longitudinal alignment with the hook 110126 of the firing driver 110114, thus “defeating” the lockout 110108 and placing the lockout bar 110128 in the unlocked state. Notably, the key 110152 comprises a length that allows the key 110152 to extend proximally from the staple cartridge 110150 to engage the cam surface 110135. As shown in FIG. 8, the point of contact between the key 110152 and the cam surface 110135 is at, or adjacent to, the pivot 110112. If the entirety of the lockout 110108 is positioned proximal to the pivot, as described above, the key 110152 comprises a length that allows the key 110152 to extend proximally from the staple cartridge 110150, past the pivot 110112, to engage the cam surface 110135 at a location proximal to the pivot 110112.

The staple cartridge 110150 may comprise a cartridge body that comprises the key 110152. The staple cartridge 110150 may comprise a retainer that comprises the key 110152. The staple cartridge 110150 may comprise a sled that comprises the key 110152. If the sled comprises the key, upon transitioning the lockout bar 110128 to the unlocked state, the firing driver 110114 is permitted to distally advance through the closure stroke portion and the firing stroke portion. As the firing driver 110114 advances distally, the sled is also advanced distally by the firing driver 110114, causing the key 110152 to progressively disengage the cam surface 110135 of the lockout bar 110128. As the key 110152 progressively disengages the cam surface 110135, the spring 110130 progressively biases the lockout bar 110128 toward the locked state. Notably, the hook 110126 of the firing driver 110114 proximally extends a length from the body 110120 that allows the firing driver 110114 to distally clear the tab 110136 before the spring 110130 repositions the lockout bar 110128 into the locked state.

Upon completion of the firing stroke, the firing driver 110114 is retracted toward its proximal position. As the firing driver 110114 is retracted, the proximal end of the hook 110126 engages the distal end of the tab 110136, which cams the lockout bar 110128 upwardly toward the unlocked state, allowing the hook 110126 to slide under the tab 110136. Once the hook 110126 proximally clears the tab 110136, the spring 110130 biases the lockout bar 110128 back into its locked state, preventing advancement of the firing driver 110114 through another closure stroke and firing stroke until another compatible staple cartridge is positioned in the end effector 110104.

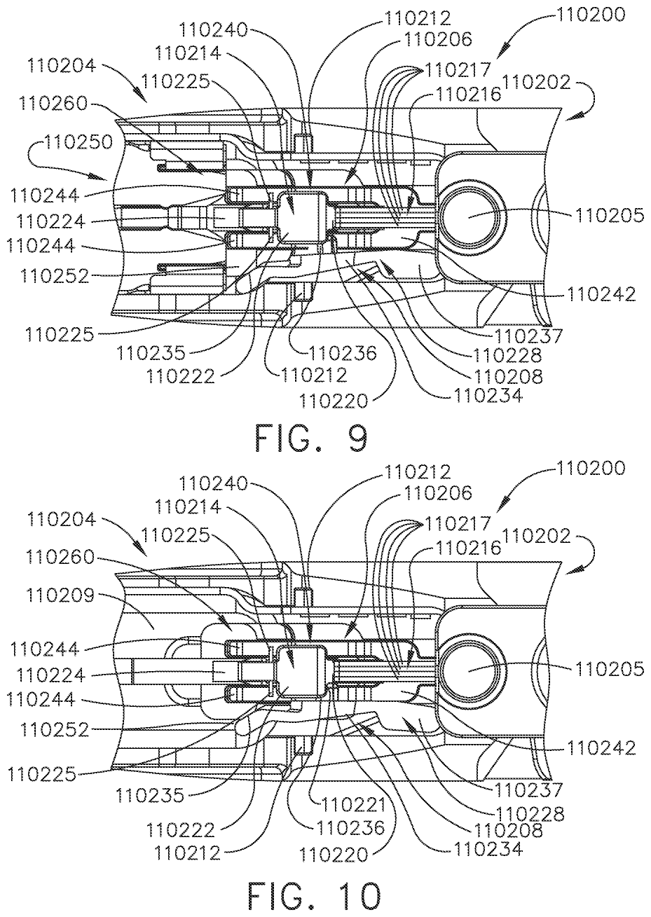

Referring now to FIGS. 9-11, a surgical stapling system 110200 is provided, according to at least one aspect of the present disclosure. The surgical stapling system 110200 comprises a shaft 110202, an end effector 110204, an articulation joint 110205, a firing actuator 110206, a closure actuator 110207, and a lockout 110208. The shaft 110202 may be similar to shaft 110002. The end effector 110204 may be similar to end effector 110004.

The end effector 110204 comprises an elongate channel 110209, an anvil 110210, and a pivot 110212 pivotably coupling the anvil 110210 to the elongate channel 110209 such that the anvil 110210 can rotate relative to the elongate channel 110209 between an open position (similar to FIG. 5) and a closed position (similar to FIG. 4). In accordance with the present disclosure, the pivot 110212 may comprise a pin. The end effector 110204 is pivotable relative to the shaft 110202 about the articulation joint 110205. The anvil 110210 comprises a tab 110215 extending from a proximal end thereof, as will be discussed in more detail below.

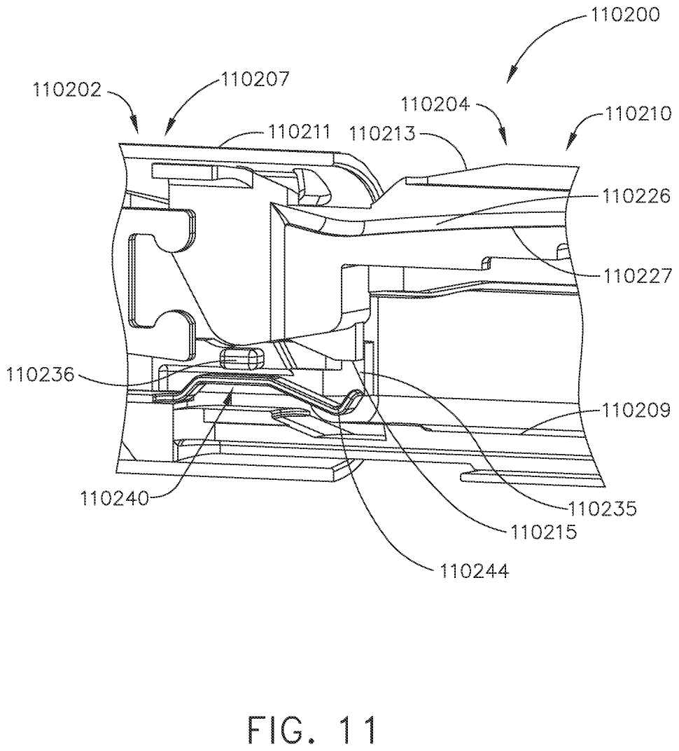

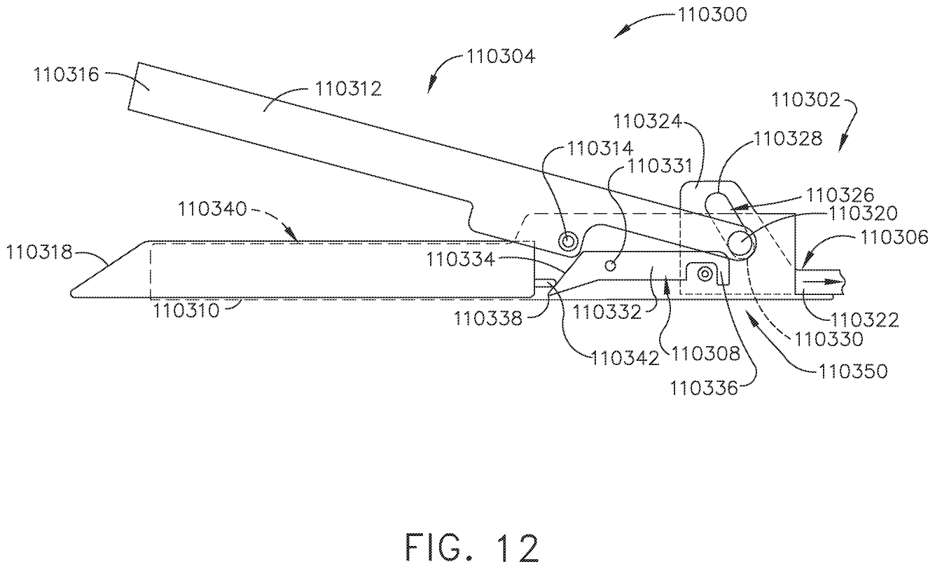

The closure actuator 110207 comprises a closure tube 110211 that can be driven using any suitable drive system, such as a motor drive system or manual drive system, to drive the closure tube 110211 from a proximal position, shown in FIG. 11, to a distal position. The anvil 110210 comprises a cam surface or ramp 110213 defined on a proximal end thereof. As the closure tube 110211 moves distally, the closure tube 110211 contacts the cam surface 110213 to cam, or rotate, the anvil 110210 from the open position toward the closed position. To transition the anvil 110210 to the open position, the closure tube 110211 may be retracted proximally and springs positioned within the end effector 110204 bias the anvil 110210 to the open position. The anvil 110210 may include a tab and the closure tube 110211 may define an aperture which engages the tab as the closure tube 110211 moves proximally to positively transition the anvil 110210 to the open position. Exemplary closure actuators and closure tubes are described in U.S. Patent Application Publication No. 2021/0059672, entitled SURGICAL INSTRUMENT WITH WIRELESS COMMUNICATION BETWEEN A CONTROL UNIT OF A ROBOTIC SYSTEM AND REMOTE SENSOR, filed Sep. 14, 2020, which is hereby incorporated by reference in its entirety herein. Various other closure actuators are contemplated by the present disclosure and are discussed elsewhere herein.

The firing actuator 110206 comprises a firing member or firing driver 110214 and a firing bar 110216 extending proximally from the firing driver 110214. The firing bar 110216 comprises a plurality of laminated strips 110217. The firing bar 110216 can be driven using any suitable drive system, such as a motor drive system, to drive the firing driver 110214 from a proximal position, shown in FIGS. 9 and 10, to a distal position. An exemplary motor drive system is described in U.S. Patent Application Publication No. 2015/0173756, entitled SURGICAL CUTTING AND STAPLING METHODS, filed Dec. 23, 2013, which is hereby incorporated by reference in its entirety herein. The firing driver 110214 comprises a firing actuator body 110220 defining an aperture 110221, a first cam 110222, a second cam (not shown, but similar to second cam 110124), a knife (not shown, but similar to knife 110125), and a lockout body 110224 pivotably coupled to the firing actuator body 110220. The lockout body 110224 comprises pins 110225 extending laterally from each side thereof.

In operation, the closure tube 110211 is moved through a closure stroke to transition the anvil 110210 from the open position to the closed position. Once the anvil 110210 has been placed in the closed position, the firing driver 110214 is moved through a firing stroke. During the firing stroke, the body 110220 of the firing driver 110114 engages and drives a sled through a staple cartridge to deploy staples therefrom while the knife cuts the stapled tissue. Furthermore, as the firing driver 110214 is advanced distally through the firing stroke, the second cam engages the elongate channel 110209 and the first cam 110222 traverses a slot 110226 defined in the anvil 110210 and engages a lower surface 110227 of the slot 110226 to control the distance, or tissue gap, between the elongate channel 110209 and the anvil 110210. The surgical stapling system 110200 may not include the closure actuator 110207. As such, the firing actuator 110206 may be configured to advance through a closure stroke portion and a firing stroke portion distal and subsequent to the closure stroke portion. During the closure stroke portion, the firing driver 110214 engages a ramp on a proximal end of the anvil 110210 to move the anvil from the open position toward the closed position. During the firing stroke portion, similar to above, the firing driver 110114 engages and drives a sled through a staple cartridge to deploy staples therefrom while also controlling the distance, or tissue gap, between the elongate channel 110209 and the anvil 110210.

The lockout 110208 includes lockout bar 110228 that includes a body 110234 defining a cam surface 110235 on its distal end thereof and a tab 110236. The lockout bar 110228 is pivotable between a locked state in which the tab 110236 is received within the aperture 110221 of the firing driver 110214 (FIG. 10) and an unlocked state in which the tab 110236 is rotated laterally out of, or displaced from, the aperture 110221 of the firing driver 110214 (FIG. 9). A proximal end 110237 of the body 110234 can be pivotably coupled to the shaft 110202. The proximal end 110237 of the body 110234 can be pivotably coupled to the elongate channel 110209. The lockout 110208 can further comprise a spring, such as a torsion spring, as an example, that biases the lockout bar 110228 toward the locked state.

In the locked state, as referenced above, the tab 110236 is received within the aperture 110221 of the firing driver 110214 such that, should a user attempt to drive the firing driver 110214 through its firing stroke, the tab 110236 would engage a proximal wall defined by the aperture 110221 and prevent distal motion of the firing driver 110214. Stated another way, in the locked state, the lockout 110208 prevents the firing driver 110214 from moving through the firing stroke. Notably, the tab 110236 engages the proximal wall defined by the aperture 110221 at a location that is laterally proximal to the pivot 110212, as will be explained in more detail below.

Furthermore, in the locked state, the body 110234 of the lockout bar 110228 is vertically aligned with the tab 110215 defined on the proximal end of the anvil 110210 such that, should a user attempt to rotate the anvil 110210 toward the closed position, the tab 110215 would engage the body 110234 of the lockout bar 110228 and prevent rotational motion thereof. Stated another way, in the locked state, the lockout bar 110228 prevents the anvil 110210 from moving to the closed position. Accordingly, in the locked state, the lockout 110208 functions to prevent both closure of the anvil 110210 and firing of the firing driver 110214. Therefore, in one aspect, the lockout 110208 can be considered a closure lockout and/or a firing lockout.

In the unlocked state, as referenced above, the tab 110236 is rotated laterally out of, or displaced from, the aperture 110221 of the firing driver 110214. In the unlocked state, the body 110234 of the lockout bar 110228 is also laterally displaced such that it is no longer vertically aligned with the tab 110215 defined on the anvil 110210 and, therefore, should a user attempt to rotate the anvil 110210 toward the closed position, the tab 110215 would bypass the body 110234 of the lockout bar 110228 during rotation of the anvil 110210. Stated another way, in the unlocked state, the lockout bar 110228 permits the anvil 110210 to move to the closed position. Furthermore, in the unlocked state, should a user attempt to drive the firing driver 110214 through the firing stroke, the tab 110236 would not engage the proximal wall defined by the aperture 110221 and prevent distal motion of the firing driver 110214. That is, in the unlocked state, the lockout 110208 permits the firing driver 110214 to move through the firing stroke.

The majority of, if not all, of the lockout 110208 is positioned proximal to the pivot 110212. In particular, the point of contact between the lockout 110208 (the tab 110236) and the firing driver 110214 (the aperture 110221) is proximal with respect to the pivot 110212. By moving the lockout 110208 proximal of the pivot 110212, space is freed up within the end effector 110204 and the lockout 110208 is moved away from the pinch point between the elongate channel 110209 and the anvil 110210. The entirety of the lockout 110108 may be positioned proximal to the pivot 110212, freeing up additional space within the end effector 110204.

To transition the lockout 110208 from the locked state to the unlocked state, a compatible staple cartridge is positioned in the elongate channel 110209. Referring to FIG. 10, no staple cartridge is removably positioned in the elongate channel 110209. Accordingly, the lockout 110208 remains in the locked state, preventing the anvil 110210 from rotating to the closed position and preventing firing driver 110214 from moving through the firing stroke. Accordingly, should a user attempt to drive the firing driver 110214 through the firing stroke, the tab 110236 would engage the proximal wall defined by the aperture 110221 at a location proximal to the pivot 110212, preventing the firing driver 110214 from moving distally. It should be understood that, should an incompatible staple cartridge that does not include key, discussed in more detail below, be removably positioned in the elongate channel 110209, the lockout 110208 would also remain in the unlocked state.

Referring now to FIG. 9, a compatible staple cartridge 110250 is removably positioned in the elongate channel 110209. The compatible staple cartridge 110250 includes a first key 110252 extending proximally therefrom. As the staple cartridge 110250 is removably positioned in the elongate channel 110209, the first key 110252 engages the cam surface 110235 of the lockout bar 110228. As the staple cartridge 110250 is moved proximally to seat the staple cartridge 110250 in the elongate channel 110209, the first key 110252 engages the cam surface 110235 and laterally rotates the tab 110236 out of the aperture 110221 and the body 110234 out of vertical alignment with the tab 110215, thus “defeating” the lockout 110208 and placing the lockout bar 110228 in the unlocked state. As shown in FIG. 10, the point of contact between the key 110252 and the cam surface 110235 is at a location distal with respect to the pivot 110212. Where the entirety of the lockout 110208 is positioned proximal to the pivot 110212, the key 110252 comprises a length that allows the key 110252 to extend proximally from the staple cartridge 110250, past the pivot 110212, to engage the cam surface 110235 at a location proximal to the pivot 110212.

The staple cartridge 110250 may comprise a cartridge body that comprises the first key 110252. The staple cartridge 110250 may comprise a retainer that comprises the first key 110252. The staple cartridge 110250 may comprise a sled that comprises the first key 110252. The lockout 110208 can be considered a compatible staple cartridge lockout as a compatible staple cartridge (i.e., a cartridge including the first key 110252) is required in order to transition the lockout bar 110228 to the unlocked state. An absent first key 110252 from a staple cartridge can be indicative of the staple cartridge being an incompatible staple cartridge.

As referenced above, the firing driver 110214 comprises a firing actuator body 110220 and a lockout body 110224 pivotably coupled to the firing actuator body 110220. The lockout body 110224 is pivotable relative to the firing actuator body 110220 between an upward, unlocked position and a downward, locked position. The firing driver 110214 can further comprise a spring that biases the lockout body 110224 toward the locked position. The end effector 110204 further comprises a spring 110240 that includes a body 110242 and a pair of hooks 110244 extending distally from the body 110242. A proximal end of the body 110242 may be mounted to the shaft 110202. The proximal end of the body 110242 may be mounted to the elongate channel 110209.

In the locked position of the lockout body 110224, the hooks 110244 of the spring 110240 “catch” the pins 110225 to prevent distal motion of the firing driver 110214. Stated another way, in the locked position of the lockout body 110224, the spring 110240 prevents the firing driver 110214 from moving through its firing stroke. In the unlocked position of the lockout body 110224, the pins 110225 are rotated upward and away from the hooks 110244 such that the lockout body 110224 and the firing driver 110214 are able to traverse past the hooks 110244 without being “caught”. Stated another way, in the unlocked position, the spring 110240 permits the firing driver 110214 to move through its firing stroke. Accordingly, the spring 110240 and the lockout body 110224 can be considered a second lockout, collectively referred to as lockout 110260. The lockout 110260 may be considered a firing lockout. The lockout 110260 may be considered a ready-to-fire lockout, as will be discussed in more detail below.

To transition the lockout body 110224 from the locked position to the unlocked position, a “ready-to-fire” staple cartridge is positioned in the elongate channel 110209. Referring now to FIG. 9, a compatible and ready-to-fire staple cartridge 110250 is removably positioned in the elongate channel 110209. As discussed above, the compatible staple cartridge 110250 includes a first key 110252 that “defeats” the first lockout 110208 to allow the anvil 110210 to move from the open position toward the closed position. Similarly, defeating the first lockout 110208 rotates the tab 110236 out of the aperture 110221 such that the firing driver 110214 is not prevented from moving through the firing stroke via the first lockout 110208.

The compatible and ready-to fire staple cartridge 110250 further includes a second key (not shown, but similar to other keys shown and described elsewhere herein) that extends proximally therefrom. As the staple cartridge 110250 is removably positioned in the elongate channel 110209, the second key slides under the lockout body 110224 to hold the lockout body 110224 in the upward, unlocked position. With the lockout body 110224 in the upward, unlocked position, as described above, the lockout body 110224 is rotated upward and away from the hooks 110244 such that the lockout body 110224 and the firing driver 110214 are able to traverse past the hooks 110244 without being “caught”. The staple cartridge 110250 can comprise a sled that comprises the second key. The spring 110240 and the lockout body 110224 can be considered a ready-to-fire lockout in that a sled with a second key in its proximal, unfired positioned is required to prevent the pins 110225 on the lockout body 110224 from falling and being “caught” by the spring 110240. An absent second key from a staple cartridge can be indicative of, for example, a staple cartridge that does not have a compatible sled (a sled that in the proximal position, but does not include a second key) or a sled with a second key not being in an unfired position (a sled being at a partially spent or spent position).

It should be understood that, in a scenario where a staple cartridge that comprises the first key 110252, but not the second key, is removably positioned in the elongate channel 110209, the first lockout 110208 would be unlocked to allow for the anvil 110210 to move to the closed position. However, despite the tab 110236 being rotated out of the aperture 110221, should a user attempt to drive the firing driver 110214 through the firing stroke, the lockout body 110224 would have no second key to maintain it in the upward, unlocked position. Therefore, the lockout body 110224 would fall to the downward locked position and the pins 110225 would be “caught” by the hooks 110244 of the spring 110240, preventing distal motion of the firing driver 110214. Accordingly, the surgical stapling system 110200 can comprise two lockouts-a first lockout (a compatible staple cartridge lockout 110208) that serves as a closure and firing lockout and a second lockout (a ready-to-fire staple cartridge lockout comprised of lockout body 110224 and spring 110240) that serves as a secondary firing lockout.

Referring now to FIG. 12, a surgical stapling system 110300 is provided, in accordance with the present disclosure. The surgical stapling system 110300 comprises a shaft 110302, an end effector 110304 extending from the shaft 110302, and a closure actuator 110306 extending through the shaft 110302. The shaft 110302 may be similar to shaft 110002. The end effector 110304 may be similar to end effector 110004.

The end effector 110304 comprises an elongate channel 110310, an anvil 110312, and a pivot 110314 pivotably coupling the anvil 110312 to the elongate channel 110310. The pivot 110314 can comprise a pin. The anvil 110312 is rotatable relative to the elongate channel 110310 between an open, locked position, where a distal tip 110316 of the anvil 110312 is rotated a first distance away from a distal tip 110318 of the elongate channel 110310, an open, unlocked position (similar to FIG. 5), where the distal tip 110316 of the anvil 110312 is rotated a second distance smaller than the first distance away from the distal tip 110318 of the elongate channel 110310, and a closed position (similar to FIG. 4) where the distal tip 110316 of the anvil 110312 is a third distance smaller than the second distance away from the distal tip 110318 of the elongate channel 110310. The anvil 110312 further comprises a pin 110320 extending from a proximal end thereof.

The closure actuator 110306 comprises an elongate bar 110322 extending along a first axis and a flange 110324 extending distally therefrom. The flange 110324 defines a slot 110326 that receives the pin 110320 and extends along a second axis that is transverse to the first axis of the elongate bar 110322. The slot 110326 comprises a first, distal end 110328 and a second, proximal end 110330 opposite the first, distal end 110328. The pin 110320 is movable along the slot 110326 between a first position, corresponding to the open, locked position of the anvil 110312, a second position, corresponding to the open, unlocked position of the anvil 110312, and a third position, corresponding to the closed position of the anvil 110312. The second position of the pin 110320 is closer to the first end 110328 of the slot 110326 than the first position of the pin 110320. The third position of the pin 110320 is closer to the first end 110328 of the slot 110326 than the second position of the pin 110320. The first position of the pin 110320 can be a position where the pin 110320 engages the second end 110330 of the slot 110326. The third position of the pin 110320 can be a position where the pin 110320 engages the first end 110328 of the slot 110326. The first position and the second position of the pin 110320 can be positions intermediate the first end 110328 of the slot 110326 and the second end 110330 of the slot 110326.

The closure actuator 110306 is movable through from a first, distal position toward a second, proximal position during a closure stroke by a drive system to move the pin 110320 along the slot 110326, and therefore, rotate the anvil 110312 from the open, unlocked position toward the closed positon. Notably, however, in the open, locked position of the anvil 110312, the closure actuator 110306 is unable to rotate the anvil 110312 toward the closed position, as explained in more detail below. The drive system that drives the closure actuator 110306 can comprise a motor-driven system. The drive system that drives the closure actuator 110306 can comprise a closure trigger and the closure actuator 110306 is manually movable according to motions provided to the closure trigger by a user.

In the open, locked position of the anvil 110312, the pin 110320 is in the first position within the slot 110326. In the first position, the flange 110324 is unable to apply a sufficient torque to the pin 110320 to cause the pin 110320 to move along the slot 110326 toward the first end 110328 thereof, and therefore, rotate the anvil 110312 away from the open, locked position. Stated another way, the closure actuator 110306 is prevented from moving proximally through its closure stroke with the pin 110320 in the first position. Accordingly, the slot 110326 of the flange 110324 and the pin 110320 collectively define a closure lockout 110350.

As shown in FIG. 12, the lockout 110350 can be positioned proximal to the pivot 110314. By moving the lockout 110350 proximal to the pivot 110314, space is freed up within the end effector 110304 and the lockout 110350 is moved away from the pinch point between the elongate channel 110310 and the anvil 110312.

The closure lockout 110350 is configurable between a locked state a locked state and an unlocked state. The locked state can correspond to the open, locked position of the anvil 110312, described above, in which the pin 110320 is positioned within the slot 110326 such that the closure actuator 110306 is prevented from moving the anvil 110312 toward the closed position. The unlocked state can correspond to the open, unlocked position of the anvil 110312, described above, in which the pin is positioned within the slot 110326 such that the closure actuator 110306 is permitted to move the anvil 110312 toward the closed position.

To transition the anvil 110312 from the open, locked position (the first position of the pin 110320) to the open, unlocked position (the second position of the pin 110320), an unlocking bar 110308 is utilized. The unlocking bar 110308 comprises a body 110332 pivotably coupled to the elongate channel 110310 with a pin 110331, a cam surface 110334 extending distally from the body 110332, and an arm 110336 extending proximally from the body 110332. The arm 110336 extends a length from the body 110332 such that, as the unlocking bar 110308 is rotated (counterclockwise as shown in FIG. 12), the arm 110336 engages the proximal end of the anvil 110312 to drive or “kick” the pin 110320 from its first position to its second position, thereby transitioning the anvil 110312 from the open, locked position to the open, unlocked position and the closure lockout 100350 from the locked state to the unlocked state.

To rotate the unlocking bar 110308, as described above, a compatible staple cartridge 110340 is positioned within the elongate channel 110310. The compatible staple cartridge 110340 comprises a key 110342 that engages the cam surface 110334 of the unlocking bar 110308 at a location that is distal to the pivot 110314 as the compatible staple cartridge 110340 is positioned in the elongate channel 110310. The staple cartridge 110340 may comprise a cartridge body that comprises the key 110342. The staple cartridge 110340 may comprise a retainer that comprises the key 110342. The staple cartridge 110340 may comprise a sled that comprises the key 110342.

The point of contact between key 110342 and the cam surface 110334 is distal to the pivot 110314. Like the closure lockout 100350, the unlocking bar 110308 can also be positioned proximal to the pivot 110314, freeing up additional space within the end effector. If the unlocking bar 110308 is also positioned proximal to the pivot 110314, the key 110342 comprises a length to extend proximally from the staple cartridge 110340, past the pivot 110314, to engage the cam surface 110334 at a location proximal to the pivot 110314.

As the staple cartridge 110340 is moved proximally to seat the staple cartridge 110340 in the elongate channel 110310, the key 110342 slides along the cam surface 110334, rotating the unlocking bar 110308. As the unlocking bar 110308 rotates, the arm 110336 of the unlocking bar 110308 engages the proximal end of the anvil 110312 to move the pin 110320 within the slot 110326 toward the second position, transitioning the anvil 110312 to the open, unlocked position and the closure lockout 110350 to the unlocked state. The unlocking bar 110308 further comprises a tip 110338 that engages the base of the elongate channel 110310 to prevent the key 110342 of the staple cartridge 110340 from over rotating the unlocking bar 110308 and moving the pin 110320 beyond its second position. The surgical stapling system 110300 can further comprise a spring that biases the tip 110338 away from the base of the elongate channel 110310 and the arm 110336 away from the pin 110320.

With the pin 110320 in the second position within the slot 110326, the closure actuator 110306 is able to apply a sufficient torque to the pin 110320 to cause the pin 110320 to move within the slot 110326 toward the first end 110328 thereof, and therefore, rotate the anvil 110312 from the open, unlocked position toward the closed position. Accordingly, in the unlocked state of the closure lockout 110350, the closure actuator 110306 is permitted to move proximally through its closure stroke to place the anvil 110312 in the closed position. Once the anvil 110312 is in the closed position, a clinician is able to perform a staple firing function to deploy the staples from the staple cartridge, as described elsewhere herein.

The surgical stapling system 110300 can further comprise a firing actuator, such as firing actuator 110106 (described above) or firing actuator 110206 (described above), that is configured to move through a firing stroke after the closure actuator 110306 has placed the anvil 110312 in the closed position to deploy the staples from the staple cartridge 110340. The surgical stapling system 110300 can further comprise a firing lockout, such as lockout 110108 or lockout 110260, that prevents the firing driver of the firing actuator from advancing through the staple cartridge 110340 unless the staple cartridge 110340 is a compatible and/or ready-to-fire staple cartridge. Accordingly, as such, the surgical stapling system 110300 may comprise two lockouts-one that prevents the anvil 110312 from rotating toward the closed position (the closure lockout 110350) and another that prevents the firing actuator from advancing through its firing stroke unless a compatible and/or ready-to-fire staple cartridge is positioned within the end effector (such as lockout 110108 or lockout 110260). The staple cartridge 110340 can comprise two keys that independently transition each lockout—the closure lockout 110350 and the firing lockout—to their respective unlocked states. The key 110342 can transition both the closure lockout 110350 and the firing lockout to their respective unlocked states.

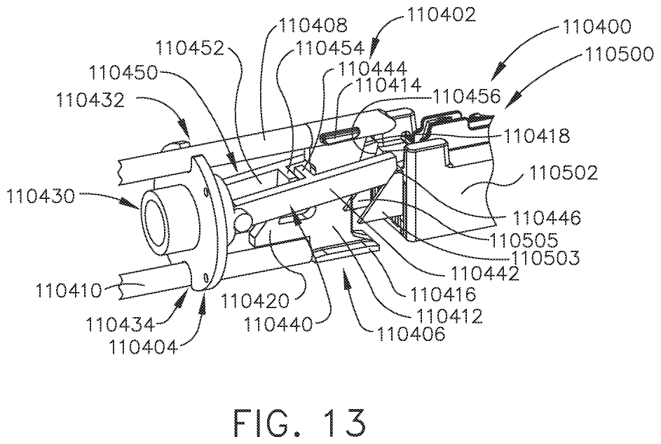

Referring now to FIGS. 13-15, a surgical stapling system 110400 is provided, in accordance with the present disclosure. The surgical stapling system 110400 comprises a firing actuator 110402 and a lockout 110404.

The firing actuator 110402 comprises a firing driver 110406, a first push rod 110408 attached at an upper end of the firing driver 110406, and a second push rod 110410 attached at a lower end of the firing driver 110406. The first push rod 110408 and the second push rod 110410 can be driven using any suitable drive system, such as a motor drive system, to drive the firing driver 110406 from a proximal position toward a distal position. An exemplary motor drive system is described in U.S. Patent Application Publication No. 2015/0173756, entitled SURGICAL CUTTING AND STAPLING METHODS, filed Dec. 23, 2013, which is hereby incorporated by reference in its entirety herein. The firing driver 110406 comprises a body 110412, a first cam 110414, a second cam 110416, a knife 110418, and a hook 110420, as will be described in more detail below.

In one aspect, the surgical stapling system 110400 further comprises an end effector (similar to end effector 110004) that comprises an elongate channel (similar to elongate channel 110007), an anvil (similar to anvil 110008), and a pivot that pivotably couples the anvil to the elongate channel about a closure axis (similar to closure axis CA). In one aspect, the pivot comprises a pin. The anvil is pivotable relative to the elongate channel about the closure axis between an open position (similar to FIG. 5) and a closed position (similar to FIG. 4). It should be noted that the end effector of the surgical stapling system 110400 is hidden in FIGS. 13-15 to help properly illustrate the firing actuator 100402 and the lockout 110404, described in more detail below.

In operation, the firing driver 110406 is movable through a stroke that includes a closure stroke portion and a firing stroke portion distal to the closure stroke portion. During the closure stroke portion, the first cam 110414 of the firing driver 110406 engages a ramp on the anvil to cam the anvil from the open position toward the closed position. During the firing stroke portion subsequent and distal to the closure stroke portion, the body 110412 of the firing driver 110406 engages and drives a sled, similar to sled 110026, through a staple cartridge, such as staple cartridge 110010, to deploy the staples therefrom while the knife 110418 cuts the stapled tissue. Furthermore, as the firing driver 110406 is advanced distally through the firing stroke portion, the second cam 110416 engages the elongate channel and the first cam 110414 engages the anvil to control the distance, or tissue gap, therebetween. In one aspect, since the firing actuator 110402 is utilized to move the anvil toward the closed position prior to deploying staples from a staple cartridge, the firing actuator 110402 can also be considered a closure actuator.

The lockout 110404 includes a flange 110430, a first lockout 110440 pivotably coupled to a first lateral side of the flange 110430, and a second lockout 110450 pivotably coupled to a second lateral side of the flange 110430. The flange 110430 defines a first recess 110432 that receives the first push rod 110408 and guides the first push rod 110408 during the closure stroke portion and the firing stroke portion. The flange 110430 further defines a second recess 110434 that receives the second push rod 110410 and guides the second push rod 110410 during the closure stroke portion and the firing stroke portion. Surgical stapling system 110400 can further comprise a shaft, similar to shaft 110002, and the flange 110430 is mounted to the shaft.

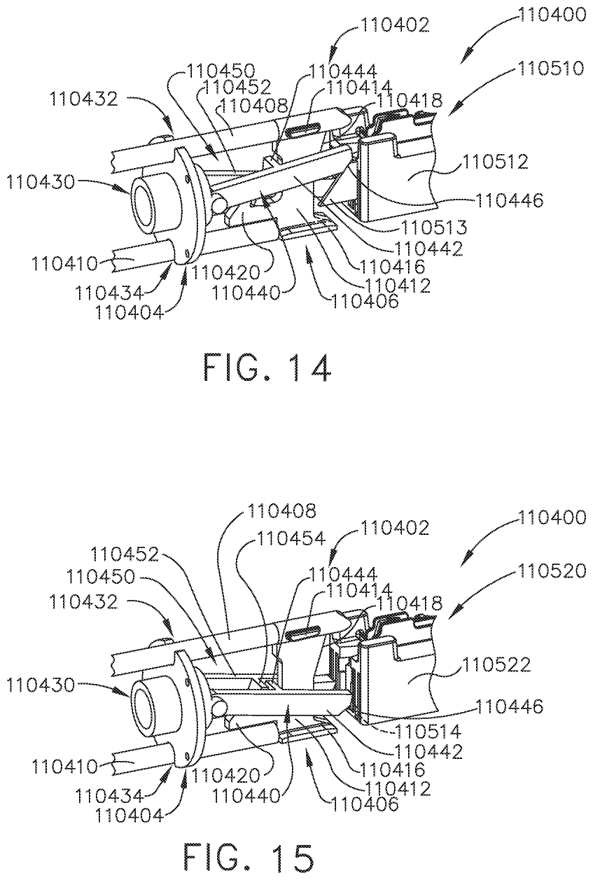

The first lockout 110440 comprises a first lockout arm 110442 pivotably coupled to the flange 110430 and a first tab 110444 that extends transversely from the first lockout arm 110442. The first lockout arm 110442 is pivotable relative to the flange 110430 between a first locked state, in which the first tab 110444 is received within the hook 110420 (FIG. 15) and a first unlocked state in which the first tab 110444 is rotated out of the hook 110420 (FIGS. 13 and 14). A spring may bias the first lockout 110440 toward the first locked state. In the first locked state, the first tab 110444 and the hook 110420 are longitudinally aligned such that, should a user attempt to drive the firing driver 110406 through the closure stroke portion and the firing stroke portion, the first tab 110444 would engage the hook 110420 at a location proximal with respect to the closure axis defined by the pivot, preventing distal motion of the firing driver 110406. The first lockout 110440 can be considered a compatible staple cartridge lockout as a compatible staple cartridge (i.e., a cartridge including a first authentication key) is required in order to transition the first lockout 110440 to the first unlocked position, as will be explained in more detail below.

Similarly, the second lockout 110450 comprises a second lockout arm 110452 pivotably coupled to the flange 110430 and a second tab 110454 that extends transversely from the second lockout arm 110452 toward the first lockout arm 110442. The second lockout 110450 is pivotable relative to the flange 110430 between a second locked state, in which the second tab 110454 is received within the hook 110420 (FIGS. 14 and 15) and a second unlocked state in which the second tab 110454 is rotated out of the hook 110420 (FIG. 13). A spring may bias the second lockout 110450 toward the second locked state. The same spring that biases the first lockout 110440 toward the first locked state may also bias the second lockout 110450 toward the second locked state. In the second locked state, the second tab 110454 and the hook 110420 are longitudinally aligned such that, should a user attempt to drive the firing driver 110406 through the closure stroke portion and the firing stroke portion, the second tab 110454 would engage the hook 110420 at a location proximal with respect to the closure axis defined by the pivot, preventing distal motion of the firing driver 110406. The second lockout 110450 can be considered a read-to-fire staple cartridge lockout as a staple cartridge that is unspent is required to transition the second lockout to the second unlocked position, as will be explained in more detail below.

A majority, if not all, of the lockout 110404 is positioned proximal with respect to the pivot between the anvil and the elongate channel. In particular, as described above, the point of contact between the firing actuator 110402 (the hook 110420) and the lockout 110404 (the first tab 110444 and/or the second tab 110454) is proximal with respect to the pivot. By moving the lockout 110404 proximal of the pivot, space is freed up within the end effector and the lockout 110404 is moved away from the pinch point between the elongate channel and the anvil. The entire lockout 110404 may be positioned proximal of the pivot, freeing up additional space within the end effector.

In use, the lockout 110404 prevents advancement of the firing driver 110406 unless a compatible and ready-to-fire staple cartridge is positioned in an elongate channel, such as elongate channel 110007, of the surgical stapling system 110400. The staple cartridge can include two discrete authentication keys where a first authentication key is configured to transition the first lockout 110440 from the first locked state to the first unlocked state and a second authentication key is configured to transition the second lockout 110450 from the second locked state to the second unlocked state. With the first lockout 110440 and the second lockout 110450 in their respective unlocked positions, the firing driver 110406 is permitted to advance through the closure stroke and the firing stroke. However, if one or both of the first lockout 110440 and the second lockout 110450 is in its respective locked position, the firing driver 110406 is prevented from advancing through the closure stroke portion and the firing stroke portion. Since the lockout 110404 prevents the firing driver 110406 from moving through the closure stroke portion and the firing stroke portion when one or both of the first lockout 110440 and the second lockout 110450 is in its respective locked position, the lockout 110404 can be considered a closure lockout and/or a firing lockout.

Referring now to FIG. 13, a staple cartridge 110500 is provided that includes a cartridge body 110502 and a sled (not shown, but similar to sled 110026). The cartridge body 110502 includes a first key 110503 and the sled comprises a second key 110505. As the staple cartridge 110500 is positioned in the elongate channel, the first key 110503 engages a cam surface 110446 on the first lockout arm 110442 to rotate the first lockout 110440 from the first locked state toward the first unlocked state. As the staple cartridge 110500 is moved proximally to seat the staple cartridge 110500 in the elongate channel, the first key 110503 clears the cam surface 110446 and then engages first lockout arm 110442 to continue camming the first lockout 110440 toward the first unlocked state, and thus, moving the first tab 110444 out of the hook 110420. The point of contact between the first key 110503 and the cam surface 110446 may be distal with respect to the pivot between the anvil and the elongate channel of the end effector. The point of contact between the first key 110503 and the cam surface 110446 may be proximal with respect to the pivot between the anvil and the elongate channel of the end effector. As such, the first key 110503 can comprise a length that allows the key 110503 to extend proximally from the staple cartridge 110500, past the pivot, to engage the cam surface 110446 at a location proximal to the pivot.

Similarly, as the staple cartridge 110500 is positioned in the elongate channel, the second key 110505 engages a cam surface 110456 on the second lockout arm 110452 to rotate the second lockout 110450 from the second locked state toward the second unlocked state. As the staple cartridge 110500 is moved proximally to seat the staple cartridge 110500 in the elongate channel, the second key 110505 clears the cam surface 110456 and then engages the second lockout arm 110452 to continue camming the second lockout 110450 toward the second unlocked state, and thus, moving the second tab 110454 out of the hook 110420. With both lockouts 110440, 110450 rotated to their respective unlocked positions, the firing driver 110406 is permitted to move through the closure stroke portion and the firing stroke portion. The point of contact between the second key 110505 and the cam surface 110456 may be distal with respect to the pivot between the anvil and the elongate channel of the end effector. The point of contact between the second key 110505 and the cam surface 110456 may be proximal with respect to the pivot between the anvil and the elongate channel of the end effector. As such, the second key 110505 can comprise a length that allows the key 110505 to extend proximally from the staple cartridge 110500, past the pivot, to engage the cam surface 110456 at a location proximal to the pivot.

Referring now to FIG. 14, the staple cartridge 110500 has been replaced with a first replacement staple cartridge 110510 that includes a cartridge body 110512 that includes a first key 100513 and a sled (not shown, but similar to sled 110026) that notably does not comprise a second key. An absent second key from the first replacement staple cartridge 110510 can be indicative of, for example, a staple cartridge that does not have a compatible sled (a sled that in the proximal position, but does not include a second key) or a sled with a second key not being in an unfired position (a sled being at a partially spent or spent position). Accordingly, when the first replacement staple cartridge 110510 is removably positioned in the elongate channel, the first lockout 110440 is transitioned to the first unlocked state (via the first key 110513, as described above) but the second lockout 110550 remains in the locked state. In such a state, the lockout 110404 prevents distal motion of the firing driver 110406. As outlined above, only one of the lockouts 110440, 110450 needs to be in its respective locked state to prevent the firing driver 110406 from moving through the closure stroke portion and the firing stroke portion.

Referring now to FIG. 15, the first replacement staple cartridge 110510 has been replaced with a second replacement staple cartridge 110520 that includes a cartridge body 110522 that notably does not comprise a first key and a sled (not shown, but similar to sled 110026) that notably does not comprise a second key. As referenced above, an absent second key from the second replacement staple cartridge 110520 can be indicative of, for example, a staple cartridge that does not have a compatible sled (a sled that in the proximal position, but does not include a second key) or a sled with a second key not being in an unfired position (a sled being at a partially spent or spent position). In addition, an absent first key from the second replacement staple cartridge 110520 can be indicative of the second replacement staple cartridge 110520 being an incompatible staple cartridge. Accordingly, when the second replacement staple cartridge 110520 is removably positioned in the elongate channel, the first lockout 110440 and the second lockout 110450 remain in their respective locked states, preventing distal motion of the firing driver 110406 through the closure stroke portion and the firing stroke portion.

When a key is formed on or otherwise provided on the sled of an otherwise compatible cartridge, persons of ordinary skill in the art will understand that the sled must be in an unfired, proximal-most position in the cartridge in order for the key to unlockingly interact with the lockout of the surgical instrument. If for example, a sled that otherwise includes an appropriate unlocking key has been moved distally out of the unfired, proximal-most position indicating that at least some of the staples have been fired and are missing (by the distal advancement of the sled), would be out of position to unlockingly engage the corresponding lockout. Such systems would thereby prevent the use of a previously fired or partially fired staple cartridge.

Many of the surgical instrument systems described herein are motivated by an electric motor; however, the surgical instrument systems described herein can be motivated in any suitable manner. The surgical instrument systems described herein can be motivated by a manually-operated trigger, for example. The motors disclosed herein may comprise a portion or portions of a robotically controlled system. Moreover, any of the end effectors and/or tool assemblies disclosed herein can be utilized with a robotic surgical instrument system. U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535, for example, discloses several examples of a robotic surgical instrument system in greater detail.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one ore more other embodiments without limitation. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

Various aspects of the subject matter described herein are set out in the following numbered examples:
Example 1—A surgical stapling system (110000, 110100, 110200, 110300, 110400) comprising an end effector (110004, 110104, 110204, 110304), a closure actuator (110106, 110207, 110306, 110402) movable from a first position toward a second position during a closure stroke, and a closure lockout (110108, 110208, 110308, 110404). The end effector comprises an elongate channel (110007, 110109, 110209, 110310), an anvil (110110, 110210, 110312), and a pivot (110112, 110212, 110314) pivotably coupling the anvil to the elongate channel. The anvil is pivotable relative to the elongate channel between an open position and a closed position. The closure actuator is configured to move the anvil toward the closed position. The closure lockout is configurable between a locked state in which the closure lockout engages the closure actuator at a location proximal with respect to the pivot to prevent the closure actuator from moving the anvil toward the closed position and an unlocked state in which the closure actuator is permitted to move the anvil toward the closed position.
Example 2—The surgical stapling system of Example 1, wherein the closure actuator comprises an elongate bar (110322) extending along a first axis and a flange (110324) extending distally from the elongate bar along a second axis transverse to the first axis.
Example 3—The surgical stapling system of Example 2, wherein the closure lockout comprises a pin (110320) extending from the anvil and a slot (110326) defined in the flange that receives the pin.
Example 4—The surgical stapling system of Example 3, wherein the open position of the anvil comprises an open, locked position. The anvil is pivotable relative to the elongate channel between the open, locked position, the closed position, and an open, unlocked position intermediate the open, locked position and the closed position.
Example 5—The surgical stapling system of Example 4, wherein the slot comprises a first end (110328) and a second end (110330). The pin is movable within the slot between a first position that is a first distance from the first end, a second position that is a second distance from the first end, and a third position that is a third distance from the first end. The first position of the pin corresponds to the open, locked position of the anvil and the locked state of the closure lockout. The second distance is less than the first distance. The second position of the pin corresponds to the open, unlocked position of the anvil and the unlocked state of the closure lockout. The third distance is less than the second distance. The third position of the pin corresponds to the closed position of the anvil.
Example 6—The surgical stapling system of Example 5, wherein the closure actuator is prevented from moving the anvil toward the closed position based on the pin being in the first position. The closure actuator is permitted to move the anvil toward the closed position based on the pin being in the second position.
Example 7—The surgical stapling system of any one of Examples 1-6, wherein the first position of the closure actuator comprises a distal position and the second position of the closure actuator comprises a proximal position.
Example 8—The surgical stapling system of any one of Examples 1-6, further comprising an unlocking bar (110308) rotatable between a first position and a second position. The unlocking bar is configured to engage the anvil to transition the closure lockout toward the unlocked state based on the unlocking bar rotating toward the second position.
Example 9—The surgical stapling system of Example 8, further comprising a staple cartridge (110340) comprising a key (110342). The key is configured to rotate the unlocking bar toward the second position to transition the closure lockout toward the unlocked state.
Example 10—The surgical stapling system of Example 8, wherein the unlocking bar is biased toward the first position.
Example 11—The surgical stapling system of Example 1, further comprising a firing actuator (110106, 110206, 110402) movable from an unfired position toward a fired position during a firing stroke and a firing lockout (110108, 110404). The firing actuator is configured to deploy staples (110021) from a staple cartridge (110340) positioned in the end effector based on the firing actuator moving toward the fired position. The firing lockout is configurable between a firing lockout state in which the firing actuator is prevented from moving through the firing stroke and an firing unlocked state in which the firing actuator is permitted to move through the firing stroke.
Example 12—The surgical stapling system of Example 1, wherein the closure actuator is further movable from the second position to a third position during a firing stroke. The closure actuator is configured to deploy staples (110021) from a staple cartridge (110010) positioned in the end effector based on the closure actuator moving from the second position toward the third position.
Example 13—The surgical stapling system of Example 1, wherein the closure actuator comprises a hook (110126, 110420). The closure lockout comprises a first tab (110136, 110444). In the locked state of the closure lockout, the first tab is longitudinally aligned with the hook to prevent the closure actuator from moving through the closure stroke. In the unlocked state of the closure lockout, the first tab is displaced from the hook to allow the closure actuator to move through the closure stroke.
Example 14—The surgical stapling system of Example 13, wherein the closure lockout further comprises a flange (110430), a first lockout arm (110442) pivotably coupled to the flange, and a second lockout arm (110452) pivotable coupled to the flange. The first tab extends transversely from the first lockout arm. The second tab (110454) extends from transversely from the second lockout arm toward the first lockout arm. In the locked state of the closure lockout, the first tab and the second tab longitudinally aligned with the hook to prevent the closure actuator from moving through the closure stroke. In the unlocked state of the closure lockout, the first tab and the second tab are displaced from the hook to allow the closure actuator to move through the closure stroke.
Example 15—The surgical stapling system of Example 1, wherein the closure lockout comprises a lockout bar (110228) pivotable between the locked state and the unlocked state. The lockout bar comprises a tab (110236). The closure actuator defines an aperture (110221). In the locked state, the tab is received in the aperture to prevent the closure actuator from moving the anvil toward the closed position. In the unlocked state, the tab is displaced from the aperture to closure actuator to move the anvil toward the closed position.
Example 16—A surgical stapling system comprising an end effector, a closure actuator movable from a first position toward a second position during a closure stroke, and a closure lockout. The end effector comprises an elongate channel, an anvil, and a pivot pivotably coupling the anvil to the elongate channel. The anvil is pivotable relative to the elongate channel between an open position and a closed position. The closure actuator is configured to move the anvil toward the closed position. The closure lockout is configurable between a locked state in which the closure lockout engages the closure actuator at a location proximal with respect to the pivot to prevent the closure actuator from moving the anvil toward the closed position and an unlocked state in which the closure actuator is permitted to move the anvil toward the closed position.
Example 17—The surgical stapling system of Example 16, wherein the closure actuator comprises an elongate bar extending along a first axis and a flange extending distally from the elongate bar along a second axis transverse to the first axis.
Example 18—The surgical stapling system of Example 17, wherein the closure lockout comprises a pin extending from the anvil and a slot defined in the flange that receives the pin.
Example 19—The surgical stapling system of Example 18, wherein the open position of the anvil comprises an open, locked position. The anvil is pivotable relative to the elongate channel between the open, locked position, the closed position, and an open, unlocked position intermediate the open, locked position and the closed position.
Example 20—The surgical stapling system of Example 19, wherein the slot comprises a first end and a second end. The pin is movable within the slot between a first position that is a first distance from the first end, a second position that is a second distance from the first end, and a third position that is a third distance from the first end. The first position of the pin corresponds to the open, locked position of the anvil and the locked state of the closure lockout. The second distance is less than the first distance. The second position of the pin corresponds to the open, unlocked position of the anvil and the unlocked state of the closure lockout. The third distance is less than the second distance. The third position of the pin corresponds to the closed position of the anvil.
Example 21—The surgical stapling system of Example 20, wherein the closure actuator is prevented from moving the anvil toward the closed position based on the pin being in the first position. The closure actuator is permitted to move the anvil toward the closed position based on the pin being in the second position.
Example 22—The surgical stapling system of Example 16, wherein the first position of the closure actuator comprises a distal position and the second position of the closure actuator comprises a proximal position.
Example 23—The surgical stapling system of Example 16, further comprising an unlocking bar rotatable between a first position and a second position. The unlocking bar is configured to engage the anvil to transition the closure lockout toward the unlocked state based on the unlocking bar rotating toward the second position.
Example 24—The surgical stapling system of Example 23, further comprising a staple cartridge comprising a key. The key is configured to rotate the unlocking bar toward the second position to transition the closure lockout toward the unlocked state.
Example 25—The surgical stapling system of Example 23, wherein the unlocking bar is biased toward the first position.
Example 26—The surgical stapling system of Example 16, further comprising a firing actuator movable from an unfired position toward a fired position during a firing stroke and a firing lockout. The firing actuator is configured to deploy staples from a staple cartridge positioned in the end effector based on the firing actuator moving toward the fired position. The firing lockout is configurable between a firing lockout state in which the firing actuator is prevented from moving through the firing stroke and an firing unlocked state in which the firing actuator is permitted to move through the firing stroke.
Example 27—The surgical stapling system of Example 16, wherein the closure actuator is further movable from the second position to a third position during a firing stroke. The closure actuator is configured to deploy staples from a staple cartridge positioned in the end effector based on the closure actuator moving from the second position toward the third position.
Example 28—The surgical stapling system of Example 16, wherein the closure actuator comprises a hook. The closure lockout comprises a first tab. In the locked state of the closure lockout, the first tab is longitudinally aligned with the hook to prevent the closure actuator from moving through the closure stroke. In the unlocked state of the closure lockout, the first tab is displaced from the hook to allow the closure actuator to move through the closure stroke.
Example 29—The surgical stapling system of Example 28, wherein the closure lockout further comprises a flange, a first lockout arm pivotably coupled to the flange, and a second lockout arm pivotable coupled to the flange. The first tab extends transversely from the first lockout arm. The second tab extends from transversely from the second lockout arm toward the first lockout arm. In the locked state of the closure lockout, the first tab and the second tab longitudinally aligned with the hook to prevent the closure actuator from moving through the closure stroke. In the unlocked state of the closure lockout, the first tab and the second tab are displaced from the hook to allow the closure actuator to move through the closure stroke.
Example 30—The surgical stapling system of Example 16, wherein the closure lockout comprises a lockout bar pivotable between the locked state and the unlocked state. The lockout bar comprises a tab. The closure actuator defines an aperture. In the locked state, the tab is received in the aperture to prevent the closure actuator from moving the anvil toward the closed position. In the unlocked state, the tab is displaced from the aperture to closure actuator to move the anvil toward the closed position.
Example 31—A surgical stapling system comprising an end effector, a firing actuator movable through a closure stroke and a firing stroke distal and subsequent to the closure stroke, and a lockout. The end effector comprises an elongate channel, an anvil, and a pivot pivotably coupling the anvil to the elongate channel. The anvil is pivotable relative to the elongate channel between an open position and a closed position. The firing actuator is configured to move the anvil toward the closed position based on the firing actuator moving through the closure stroke. The firing actuator is configured to deploy staples from a staple cartridge positioned in the end effector based on the firing actuator moving through the firing stroke. The lockout is configurable between a locked state in which the lockout engages the firing actuator proximal with respect to the pivot to prevent the firing actuator from moving through the closure stroke and the firing stroke and an unlocked state in which firing actuator is allowed to move through the closure stroke and the firing stroke.
Example 32—The surgical stapling system of Example 31, wherein the firing actuator comprises a hook. The lockout comprises a first tab. In the locked state of the lockout, the first tab is longitudinally aligned with the hook to prevent the firing actuator from moving through the closure stroke and the firing stroke. In the unlocked state of the lockout, the first tab is displaced from the hook to allow the firing actuator to move through the closure stroke and the firing stroke.
Example 33—The surgical stapling system of Example 32, wherein the lockout further comprises a flange, a first lockout arm pivotably coupled to the flange, and a second lockout arm pivotable coupled to the flange. A first tab extends transversely from the first lockout arm. A second tab extends from transversely from the second lockout arm toward the first lockout arm. In the locked state of the lockout, at least one of the first tab or the second tab is longitudinally aligned with the hook to prevent the firing actuator from moving through the closure stroke and the firing stroke. In the unlocked state of the lockout, the first tab and the second tab are displaced from the hook to allow the firing actuator to move through the closure stroke and the firing stroke.
Example 34—A surgical stapling system comprising an end effector, a closure actuator movable from a first position toward a second position during a closure stroke, a firing actuator movable from an unfired position toward a fired position during a firing stroke, a closure lockout, and a firing lockout. The end effector comprises an elongate channel, an anvil, and a pivot pivotably coupling the anvil to the elongate channel. The anvil is pivotable relative to the elongate channel between an open position and a closed position. The closure actuator is configured to move the anvil toward the closed position; The firing actuator comprises a knife configured to cut tissue captured between the elongate channel and the anvil. The closure lockout is positioned proximal with respect to the pivot. The closure lockout is configurable between a first locked state in which the closure actuator is prevented from moving the anvil toward the closed position and a first unlocked state in which the closure actuator is permitted to move the anvil toward the closed position. The firing lockout is configurable between a second locked state in which the firing actuator is prevented from moving toward the fired position and a second unlocked state in which the firing actuator is permitted to move toward the fired position.
Example 35—The surgical stapling system of Example 34, wherein the end effector further comprises a staple cartridge comprising a first key configured to engage the closure lockout to place the closure lockout into the first unlocked state and a second key configured to engage the firing lockout to place the firing lockout into the second unlocked state.

The entire disclosures of U.S. Pat. No. 11,589,865, entitled METHODS FOR CONTROLLING A POWERED SURGICAL STAPLER THAT HAS SEPARATE ROTARY CLOSURE AND FIRING SYSTEMS, which issued on Feb. 28, 2023, U.S. Pat. No. 6,978,921, entitled SURGICAL STAPLING INSTRUMENT INCORPORATING AN E-BEAM FIRING MECHANISM, which issued on Dec. 27, 2005, U.S. Pat. No. 10,213,203, entitled STAPLE CARTRIDGE ASSEMBLY WITHOUT A BOTTOM COVER, which issued on Feb. 26, 2019, U.S. Pat. No. 10,945,727, entitled STAPLE CARTRIDGE WITH DEFORMABLE DRIVER RETENTION FEATURES, which issued on Mar. 16, 2021, U.S. Pat. No. 11,234,698, entitled STAPLING SYSTEM COMPRISING A CLAMP LOCKOUT AND A FIRING LOCKOUT, which issued on Feb. 1, 2022, U.S. Pat. No. 11,540,826, entitled SURGICAL STAPLER END EFFECTOR SLED HAVING CARTRIDGE WALL SUPPORT FEATURE, which issued on Jan. 3, 2023, U.S. Pat. No. 10,299,792, entitled FASTENER CARTRIDGE COMPRISING NON-UNIFORM FASTENERS, which issued on May 28, 2019, U.S. Pat. No. 8,540,133, entitled STAPLE CARTRIDGE, which issued on Sep. 24, 2013, U.S. Pat. No. 9,788,835, entitled DEVICES AND METHODS FOR FACILITATING EJECTION OF SURGICAL FASTENERS FROM CARTRIDGES, which issued on Oct. 17, 2017, U.S. Pat. No. 10,105,142, entitled SURGICAL STAPLER WITH PLURALITY OF CUTTING ELEMENTS, which issued on Oct. 23, 2018, U.S. Pat. No. 10,537,324, entitled STEPPED STAPLE CARTRIDGE WITH ASYMMETRICAL STAPLES, which issued on Jan. 21, 2020, U.S. Pat. No. 7,669,746, entitled STAPLE CARTRIDGES FOR FORMING STAPLES HAVING DIFFERING FORMED STAPLE HEIGHTS, which issued on Mar. 2, 2010, U.S. Pat. No. 8,123,100, entitled SURGICAL STAPLING INSTRUMENTS INCLUDING A CARTRIDGE HAVING MULTIPLE STAPLE SIZES, which issued on Feb. 28, 2012, U.S. Pat. No. 7,407,075, entitled STAPLE CARTRIDGE HAVING MULTIPLE STAPLE SIZES FOR A SURGICAL STAPLING INSTRUMENT, which issued on Aug. 5, 2008, U.S. Pat. No. 10,085,749, entitled SURGICAL APPARATUS WITH CONDUCTOR STRAIN RELIEF, which issued on Oct. 2, 2018, U.S. Pat. No. 10,765,427, entitled METHOD FOR ARTICULATING A SURGICAL INSTRUMENT, which issued on Sep. 8, 2020, U.S. Pat. No. 11,291,445, entitled SURGICAL STAPLE CARTRIDGES WITH INTEGRAL AUTHENTICATION KEYS, which issued on Apr. 5, 2022, U.S. Pat. No. 8,864,007, entitled IMPLANTABLE FASTENER CARTRIDGE HAVING A NON-UNIFORM ARRANGEMENT, which issued on Oct. 21, 2014, U.S. Pat. No. 11,490,890, entitled COMPRESSIBLE NON-FIBROUS ADJUNCTS, which issued on Nov. 8, 2022, U.S. Pat. No. 10,952,724, entitled THREE DIMENSIONAL ADJUNCTS, which issued on Mar. 23, 2021, U.S. Pat. No. 9,770,245, entitled LAYER ARRANGEMENTS FOR SURGICAL STAPLE CARTRIDGES, which issued on Sep. 26, 2017, U.S. Pat. No. 10,123,798, entitled TISSUE THICKNESS COMPENSATOR COMPRISING CONTROLLED RELEASE AND EXPANSION, which issued on Nov. 13, 2018, U.S. Pat. No. 10,166,023, entitled METHOD OF APPLYING A BUTTRESS TO A SURGICAL STAPLER END EFFECTOR, which issued on Jan. 1, 2019, U.S. Pat. No. 11,207,065, entitled METHOD FOR FABRICATING SURGICAL STAPLER ANVILS, which issued on Dec. 28, 2021, U.S. Pat. No. 8,141,762, entitled SURGICAL STAPLER COMPRISING A STAPLE POCKET, which issued on Mar. 27, 2012, U.S. Pat. No. 8,876,857, entitled END EFFECTOR WITH REDUNDANT CLOSING MECHANISMS, which issued on Nov. 4, 2014, U.S. Pat. No. 9,629,631, entitled COMPOSITE DRIVE BEAM FOR SURGICAL STAPLING, which issued on Apr. 25, 2017, U.S. Patent Application Publication No. 2022/0346858, entitled METHOD FOR OPERATING A SURGICAL INSTRUMENT INCLUDING SEGMENTED ELECTRODES, which published on Nov. 3, 2022, U.S. Patent Application Publication No. 2022/0304680, entitled DRIVERS FOR FASTENER CARTRIDGE ASSEMBLIES HAVING ROTARY DRIVE SCREWS, which published on Sep. 29, 2022, U.S. Patent Application Publication No. 2022/0304679, entitled METHOD OF USING A POWERED STAPLING DEVICE, which published on Sep. 29, 2022, U.S. Patent Publication No. 2019/0298350, entitled METHODS FOR CONTROLLING A POWERED SURGICAL STAPLER THAT HAS SEPARATE ROTARY CLOSURE AND FIRING SYSTEMS, which published on Oct. 3, 2019, U.S. Patent Application Publication No. 2017/0367695, entitled STAPLE CARTRIDGE COMPRISING WIRE STAPLES AND STAMPED STAPLES, which published on Dec. 28, 2017, U.S. Patent Application Publication No. 2015/0134077, entitled SEALING MATERIALS FOR USE IN SURGICAL STAPLING, which published on May 14, 2015, U.S. Patent Application Publication No. 2018/0168615, entitled METHOD OF DEFORMING STAPLES FROM TWO DIFFERENT TYPES OF STAPLE CARTRIDGES WITH THE SAME SURGICAL STAPLING INSTRUMENT, which published on Jun. 21, 2018, U.S. Patent Application Publication No. 2018/0132849, entitled STAPLE FORMING POCKET CONFIGURATIONS FOR CIRCULAR SURGICAL STAPLER ANVIL, which published on May 17, 2018, U.S. Patent Application Publication No. 2018/0168613, entitled SURGICAL INSTRUMENTS WITH JAWS THAT ARE PIVOTABLE ABOUT A FIXED AXIS AND INCLUDE SEPARATE AND DISTINCT CLOSURE AND FIRING SYSTEMS, which published on Jun. 21, 2018, U.S. Patent Application Publication No. 2017/0319205, entitled POWERED END EFFECTOR ASSEMBLY WITH PIVOTABLE CHANNEL, which published on Nov. 9, 2017, U.S. Patent Application Publication No. 2014/0001231, entitled FIRING SYSTEM LOCKOUT ARRANGEMENTS FOR SURGICAL INSTRUMENTS, which published on Jan. 2, 2014, U.S. Patent Application Publication No. 2016/0095596, entitled APPARATUS FOR ENDOSCOPIC PROCEDURES, which published on Apr. 7, 2016, U.S. Patent Application Publication No. 2015/0297199, entitled ADAPTER ASSEMBLY WITH GIMBAL FOR INTERCONNECTING ELECTROMECHANICAL SURGICAL DEVICES AND SURGICAL LOADING UNITS, AND SURGICAL SYSTEMS THEREOF, which published on Oct. 22, 2015, U.S. Patent Application Publication No. 2022/0031351, entitled SURGICAL INSTRUMENTS WITH DIFFERENT ARTICULATION JOINT ARRANGEMENTS FOR ACCOMMODATING FLEXIBLE ACTUATORS, which published on Feb. 3, 2022, U.S. Patent Application Publication No. 2022/0031320, entitled SURGICAL INSTRUMENTS WITH FLEXIBLE FIRING MEMBER ACTUATOR CONSTRAINT ARRANGEMENTS, which published on Feb. 3, 2022, U.S. Patent Application Publication No. 2023/0119119, entitled CABLE-DRIVEN ACTUATION SYSTEM FOR ROBOTIC SURGICAL TOOL ATTACHMENT, which published on Apr. 20, 2023, International Patent Publication No. WO2018/071497, entitled STAPLER CARTRIDGE WITH AN INTEGRAL KNIFE, which published on Apr. 18, 2018, International Patent Publication No. WO2018/049211, entitled WRIST ARCHITECTURE, which published on Mar. 15, 2018, U.S. Pat. No. 11,298,129, entitled METHOD FOR PROVIDING AN AUTHENTICATION LOCKOUT IN A SURGICAL STAPLER WITH A REPLACEABLE CARTRIDGE, which issued on Apr. 12, 2022, U.S. Pat. No. 10,898,183, entitled ROBOTIC SURGICAL INSTRUMENT WITH CLOSED LOOP FEEDBACK TECHNIQUES FOR ADVANCEMENT OF CLOSURE MEMBER DURING FIRING, which issued on Jan. 26, 2021, U.S. Pat. No. 5,485,947, entitled LINEAR STAPLING MECHANISM WITH CUTTING MEANS, which issued on Jan. 23, 1996, International Patent Publication No. WO2018/049206, entitled STAPLER RELOAD DETECTION AND IDENTIFICATION, which published on Mar. 15, 2018, U.S. Patent Application Publication No. 2016/0249920, entitled Surgical fastener applying apparatus, which published on Sep. 1, 2016, U.S. Design Pat. No. D974,560, entitled STAPLE CARTRIDGE, which issued on Jan. 3, 2023, U.S. Design Pat. No. D967,421, entitled STAPLE CARTRIDGE, which issued on Oct. 18, 2022, U.S. Design Pat. No. D933,220, entitled BUTTRESS ASSEMBLY FOR A SURGICAL STAPLER, which issued on Oct. 12, 2021, U.S. Pat. No. 9,839,420, entitled TISSUE THICKNESS COMPENSATOR COMPRISING AT LEAST ONE MEDICAMENT, which issued on Dec. 12, 2017, U.S. Pat. No. 10,588,623, entitled ADHESIVE FILM LAMINATE, which issued on Mar. 17, 2020, U.S. Pat. No. 8,499,992, entitled DEVICE AND METHOD FOR CONTROLLING COMPRESSION OF TISSUE, which issued on Aug. 6, 2013, U.S. Patent Application Publication No. 2022/0378427, entitled STAPLING INSTRUMENT COMPRISING JAW MOUNTS, which published on Dec. 1, 2022, U.S. Pat. No. 10,349,939, entitled METHOD OF APPLYING A BUTTRESS TO A SURGICAL STAPLER, which issued on Jul. 16, 2019, U.S. Pat. No. 9,386,988, entitled RETAINER ASSEMBLY INCLUDING A TISSUE THICKNESS COMPENSATOR, which issued on Jul. 12, 2016, U.S. U.S. Pat. No. 9,072,535, entitled SURGICAL STAPLING INSTRUMENTS WITH ROTATABLE STAPLE DEPLOYMENT ARRANGEMENTS, which issued on Jul. 7, 2015, and U.S. Pat. No. 9,844,369, entitled, SURGICAL END EFFECTORS WITH FIRING ELEMENT MONITORING ARRANGEMENTS, which issued on Dec. 19, 2017 are incorporated by reference herein.

The entire disclosures of:
U.S. Pat. No. 5,403,312, entitled ELECTROSURGICAL HEMOSTATIC DEVICE, which issued on Apr. 4, 1995;
U.S. Pat. No. 7,000,818, entitled SURGICAL STAPLING INSTRUMENT HAVING SEPARATE DISTINCT CLOSING AND FIRING SYSTEMS, which issued on Feb. 21, 2006;
U.S. Pat. No. 7,422,139, entitled MOTOR-DRIVEN SURGICAL CUTTING AND FASTENING INSTRUMENT WITH TACTILE POSITION FEEDBACK, which issued on Sep. 9, 2008;
U.S. Pat. No. 7,464,849, entitled ELECTRO-MECHANICAL SURGICAL INSTRUMENT WITH CLOSURE SYSTEM AND ANVIL ALIGNMENT COMPONENTS, which issued on Dec. 16, 2008;
U.S. Pat. No. 7,670,334, entitled SURGICAL INSTRUMENT HAVING AN ARTICULATING END EFFECTOR, which issued on Mar. 2, 2010;
U.S. Pat. No. 7,753,245, entitled SURGICAL STAPLING INSTRUMENTS, which issued on Jul. 13, 2010;
U.S. Pat. No. 8,393,514, entitled SELECTIVELY ORIENTABLE IMPLANTABLE FASTENER CARTRIDGE, which issued on Mar. 12, 2013;
U.S. patent application Ser. No. 11/343,803, entitled SURGICAL INSTRUMENT HAVING RECORDING CAPABILITIES, now U.S. Pat. No. 7,845,537;
U.S. patent application Ser. No. 12/031,573, entitled SURGICAL CUTTING AND FASTENING INSTRUMENT HAVING RF ELECTRODES, filed Feb. 14, 2008;
U.S. patent application Ser. No. 12/031,873, entitled END EFFECTORS FOR A SURGICAL CUTTING AND STAPLING INSTRUMENT, filed Feb. 15, 2008, now U.S. Pat. No. 7,980,443;
U.S. patent application Ser. No. 12/235,782, entitled MOTOR-DRIVEN SURGICAL CUTTING INSTRUMENT, now U.S. Pat. No. 8,210,411;
U.S. patent application Ser. No. 12/235,972, entitled MOTORIZED SURGICAL INSTRUMENT, now U.S. Pat. No. 9,050,083.
U.S. patent application Ser. No. 12/249,117, entitled POWERED SURGICAL CUTTING AND STAPLING APPARATUS WITH MANUALLY RETRACTABLE FIRING SYSTEM, now U.S. Pat. No. 8,608,045;
U.S. patent application Ser. No. 12/647,100, entitled MOTOR-DRIVEN SURGICAL CUTTING INSTRUMENT WITH ELECTRIC ACTUATOR DIRECTIONAL CONTROL ASSEMBLY, filed Dec. 24, 2009, now U.S. Pat. No. 8,220,688;
U.S. patent application Ser. No. 12/893,461, entitled STAPLE CARTRIDGE, filed Sep. 29, 2012, now U.S. Pat. No. 8,733,613;
U.S. patent application Ser. No. 13/036,647, entitled SURGICAL STAPLING INSTRUMENT, filed Feb. 28, 2011, now U.S. Pat. No. 8,561,870;
U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535;
U.S. patent application Ser. No. 13/524,049, entitled ARTICULATABLE SURGICAL INSTRUMENT COMPRISING A FIRING DRIVE, filed on Jun. 15, 2012, now U.S. Pat. No. 9,101,358;
U.S. patent application Ser. No. 13/800,025, entitled STAPLE CARTRIDGE TISSUE THICKNESS SENSOR SYSTEM, filed on Mar. 13, 2013, now U.S. Pat. No. 9,345,481;
U.S. patent application Ser. No. 13/800,067, entitled STAPLE CARTRIDGE TISSUE THICKNESS SENSOR SYSTEM, filed on Mar. 13, 2013, now U.S. Patent Application Publication No. 2014/0263552;
U.S. Patent Application Publication No. 2007/0175955, entitled SURGICAL CUTTING AND FASTENING INSTRUMENT WITH CLOSURE TRIGGER LOCKING MECHANISM, filed Jan. 31, 2006; and
U.S. Patent Application Publication No. 2010/0264194, entitled SURGICAL STAPLING INSTRUMENT WITH AN ARTICULATABLE END EFFECTOR, filed Apr. 22, 2010, now U.S. Pat. No. 8,308,040, are hereby incorporated by reference herein.

While several configurations have been described, additional modifications are within the scope of the present disclosure, which is intended to cover any variations, uses, or adaptations of the disclosed configurations using its general principles.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

It is worthy to note that any reference to “one aspect,” “an aspect,” “an exemplification,” “one exemplification,” and the like means that a particular feature, structure, or characteristic described in connection with the aspect is included in one aspect. Thus, appearances of the phrases “in one aspect,” “in an aspect,” “in an exemplification,” and “in one exemplification” in various places throughout the specification are not necessarily all referring to the same aspect. The particular features, structures or characteristics may be combined in any suitable manner in various aspects.

It is worthy to note that any reference numbers included in the appended claims are used to reference exemplary embodiments/elements described in the present disclosure. Accordingly, any such reference numbers are not meant to limit the scope of the subject matter recited in the appended claims.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1-15. (canceled)

16. A surgical stapling system, comprising:
an end effector, comprising:
an elongate channel;
an anvil; and
a pivot pivotably coupling the anvil to the elongate channel about a first pivot axis, wherein the anvil is pivotable relative to the elongate channel between an open position and a closed position;
a closure actuator movable from a first position toward a second position during a closure stroke, wherein the closure actuator is configured to move the anvil toward the closed position; and
a closure lockout pivotable about a second pivot axis between:
a locked state in which the closure lockout engages the closure actuator at a location proximal with respect to the pivot to prevent the closure actuator from moving the anvil toward the closed position; and
an unlocked state in which the closure actuator is permitted to move the anvil toward the closed position, wherein the second pivot axis is proximal to the first pivot axis.

17. The surgical stapling system of claim 16, wherein the closure actuator comprises:
an elongate bar extending along a first axis; and
a flange extending distally from the elongate bar along a second axis transverse to the first axis.

18. The surgical stapling system of claim 17, wherein the closure lockout comprises:
a pin extending from the anvil; and
a slot defined in the flange that receives the pin.

19. The surgical stapling system of claim 18, wherein the open position of the anvil comprises an open, locked position, and wherein the anvil is pivotable relative to the elongate channel between:
the open, locked position;
the closed position; and
an open, unlocked position intermediate the open, locked position and the closed position.

20. The surgical stapling system of claim 19, wherein the slot comprises a first end and a second end, and wherein the pin is movable within the slot between:
a first position that is a first distance from the first end, wherein the first position of the pin corresponds to the open, locked position of the anvil and the locked state of the closure lockout;
a second position that is a second distance from the first end, wherein the second distance is less than the first distance, and wherein the second position of the pin corresponds to the open, unlocked position of the anvil and the unlocked state of the closure lockout; and
a third position that is a third distance from the first end, wherein the third distance is less than the second distance, and wherein the third position of the pin corresponds to the closed position of the anvil.

21. The surgical stapling system of claim 20, wherein the closure actuator is prevented from moving the anvil toward the closed position based on the pin being in the first position, and wherein the closure actuator is permitted to move the anvil toward the closed position based on the pin being in the second position.

22. The surgical stapling system of claim 16, wherein the first position of the closure actuator comprises a distal position, and wherein the second position of the closure actuator comprises a proximal position.

23. The surgical stapling system of claim 16, further comprising an unlocking bar rotatable between a first position and a second position, wherein the unlocking bar is configured to engage the anvil to transition the closure lockout toward the unlocked state based on the unlocking bar rotating toward the second position.

24. The surgical stapling system of claim 23, further comprising a staple cartridge comprising a key, wherein the key is configured to rotate the unlocking bar toward the second position to transition the closure lockout toward the unlocked state.

25. The surgical stapling system of claim 23, wherein the unlocking bar is biased toward the first position.

26. The surgical stapling system of claim 16, further comprising:
a firing actuator movable from an unfired position toward a fired position during a firing stroke, wherein the firing actuator is configured to deploy staples from a staple cartridge positioned in the end effector based on the firing actuator moving toward the fired position; and
a firing lockout, configurable between:
a firing lockout state in which the firing actuator is prevented from moving through the firing stroke; and
a firing unlocked state in which the firing actuator is permitted to move through the firing stroke.

27. The surgical stapling system of claim 16, wherein the closure actuator is further movable from the second position to a third position during a firing stroke, wherein closure actuator is configured to deploy staples from a staple cartridge positioned in the end effector based on the closure actuator moving from the second position toward the third position.

28. The surgical stapling system of claim 16, wherein the closure actuator comprises a hook, wherein the closure lockout comprises a first tab, and wherein:
in the locked state of the closure lockout, the first tab is longitudinally aligned with the hook to prevent the closure actuator from moving through the closure stroke; and
in the unlocked state of the closure lockout, the first tab is displaced from the hook to allow the closure actuator to move through the closure stroke.

29. The surgical stapling system of claim 28, wherein said closure lockout further comprises:
a flange;
a first lockout a first lockout arm pivotably coupled to said flange about said second pivot axis, wherein said first tab extends transversely from said first lockout arm; and
a second lockout a second lockout arm pivotable coupled to said flange about said second pivot axis, wherein a second tab extends from transversely from said second lockout arm toward said first lockout arm;
wherein:
in said locked state of said closure lockout, said first tab and said second tab longitudinally aligned with said hook to prevent said closure actuator from moving through said closure stroke; and
in said unlocked state of said closure lockout, said first tab and said second tab are displaced from said hook to allow said closure actuator to move through said closure stroke.

30. The surgical stapling system of claim 16, wherein the closure lockout comprises a lockout bar pivotable between the locked state and the unlocked state, wherein the lockout bar comprises a tab, wherein the closure actuator defines an aperture, and wherein:
in the locked state, the tab is received in the aperture to prevent the closure actuator from moving the anvil toward the closed position; and
in the unlocked state, the tab is displaced from the aperture to allow the closure actuator to move the anvil toward the closed position.

31. A surgical stapling system, comprising:
an end effector, comprising:
an elongate channel;
an anvil; and
a pivot pivotably coupling the anvil to the elongate channel about a first pivot axis, wherein the anvil is pivotable relative to the elongate channel between an open position and a closed position;
a firing actuator movable through a closure stroke and a firing stroke distal and subsequent to the closure stroke, wherein the firing actuator is configured to move the anvil toward the closed position based on the firing actuator moving through the closure stroke, and wherein the firing actuator is configured to deploy staples from a staple cartridge positioned in the end effector based on the firing actuator moving through the firing stroke; and
a lockout pivotable about a second pivot axis between:
a locked state in which the lockout engages the firing actuator proximal with respect to the pivot to prevent the firing actuator from moving through the closure stroke and the firing stroke; and
an unlocked state in which firing actuator is allowed to move through the closure stroke and the firing stroke, wherein the second pivot axis is proximal to the first pivot axis.

32. The surgical stapling system of claim 31, wherein the firing actuator comprises a hook, wherein the lockout comprises a first tab, and wherein:
in the locked state of the lockout, the first tab is longitudinally aligned with the hook to prevent the firing actuator from moving through the closure stroke and the firing stroke; and
in the unlocked state of the lockout, the first tab is displaced from the hook to allow the firing actuator to move through the closure stroke and the firing stroke.

33. The surgical stapling system of claim 32, wherein the lockout further comprises:
a flange;
a first lockout comprising a first lockout arm pivotably coupled to the flange about said second pivot axis, wherein the first tab extends transversely from the first lockout arm; and
a second lockout comprising a second lockout arm pivotable coupled to the flange about said second pivot axis, wherein a second tab extends from transversely from the second lockout arm toward the first lockout arm;
wherein:
in the locked state of the lockout, the first tab, the second tab, or both the first tab and the second tab longitudinally align with the hook to prevent the firing actuator from moving through the closure stroke and the firing stroke; and
in the unlocked state of the lockout, the first tab and the second tab are displaced from the hook to allow the firing actuator to move through the closure stroke and the firing stroke.

34. A surgical stapling system, comprising:
an end effector, comprising:
an elongate channel;
an anvil; and
a pivot pivotably coupling the anvil to the elongate channel about a first pivot axis, wherein the anvil is pivotable relative to the elongate channel between an open position and a closed position;
a closure actuator movable from a first position toward a second position during a closure stroke, wherein the closure actuator is configured to move the anvil toward the closed position;
a firing actuator movable from an unfired position toward a fired position during a firing stroke, wherein the firing actuator comprises a knife configured to cut tissue captured between the elongate channel and the anvil;
a closure lockout positioned proximal with respect to the pivot, wherein the closure lockout is pivotable about a second pivot axis between:
a first locked state in which the closure actuator is prevented from moving the anvil toward the closed position; and
a first unlocked state in which the closure actuator is permitted to move the anvil toward the closed position, wherein the second pivot axis is proximal to the first pivot axis; and
a firing lockout, configurable between:
a second locked state in which the firing actuator is prevented from moving toward the fired position; and
a second unlocked state in which the firing actuator is permitted to move toward the fired position.

35. The surgical stapling system of claim 34, wherein the end effector further comprises a staple cartridge, comprising:
a first key configured to engage the closure lockout to place the closure lockout into the first unlocked state; and
a second key configured to engage the firing lockout to place the firing lockout into the second unlocked state.
